(19) United States
(12) Patent Application Publication
(10) Pub. No.: US 2024/0158425 A1
(43) Pub. Date: May 16, 2024

(54) MODIFIED ADENINES

(71) Applicant: NUCLERA LTD, Cambridge (GB)

(72) Inventors: Gordon Ross MCINROY, Cambridge (GB); Martin Edward FOX, Cambridge (GB); Puneet SRIVASTAVA, Cambridge (GB); Michal Robert MATUSZEWSKI, Cambridge (GB)

(21) Appl. No.: 18/277,474

(22) PCT Filed: Feb. 21, 2022
(86) PCT No.: PCT/GB2022/050462

§ 371 Date: Aug. 16, 2023

(30) Foreign Application Priority Data
Feb. 19, 2021 (GB) 2102379.1

(51) Int. Cl. C07F 9/6558; C07H 19/20; C07H 21/04; C12P 19/34

(52) U.S. Cl. CPC C07F 9/65586; C07H 19/20; C07H 21/04; C12P 19/34

(57) ABSTRACT

The present invention relates to a compound according to Formula (1a) or (1b):

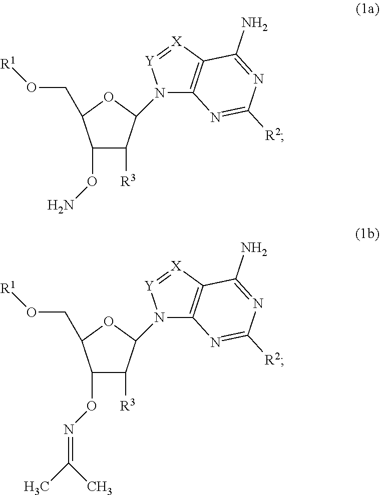

wherein R¹; R²; R³ X and Y are defined herein, and their use in methods of nucleic acid synthesis.

### FIELD OF THE INVENTION

The invention relates to modified purine nucleotides having an electron withdrawing groups on the purine ring. The Electron withdrawing groups can be at the 2-position, 7-position or 8-position. The invention also relates to a method of nucleic acid synthesis to produce oligonucleotides containing said modified purine nucleotide. The invention further relates to a kit comprising the modified purine, a terminal transferase enzyme and optionally a salt.

### BACKGROUND TO THE INVENTION

Nucleic acid synthesis is vital to modern biotechnology. The rapid pace of development in the biotechnology arena has been made possible by the scientific community's ability to artificially synthesise DNA, RNA and proteins.

Artificial DNA synthesis allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. Despite being a mature technology, it is highly challenging to synthesise a DNA strand greater than 200 nucleotides in length in viable yield, and most DNA synthesis companies only offer up to 120 nucleotides routinely. In comparison, an average protein-coding gene is of the order of 2000-3000 contiguous nucleotides, a chromosome is at least a million contiguous nucleotides in length and an average eukaryotic genome numbers in the billions of nucleotides. In order to prepare nucleic acid strands thousands of base pairs in length, all major gene synthesis companies today rely on variations of a ‘synthesise and stitch’ technique, where overlapping 40-60-mer fragments are synthesised and stitched together by enzymatic copying and extension. Current methods generally allow up to 3 kb in length for routine production.

The reason DNA cannot be routinely synthesised beyond 120-200 nucleotides at a time is due to the current methodology for generating DNA, which uses synthetic chemistry (i.e., phosphoramidite technology) to couple a nucleotide one at a time to make DNA. Even if the efficiency of each nucleotide-coupling step is 99% efficient, it is mathematically impossible to synthesise DNA longer than 200 nucleotides in acceptable yields. The Venter Institute illustrated this laborious process by spending 4 years and 20 million USD to synthesise the relatively small genome of a bacterium.

Known methods of DNA sequencing use template-dependent DNA polymerases to add 3′-reversibly terminated nucleotides to a growing double-stranded substrate. In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

Various attempts have been made to use a terminal deoxynucleotidyl transferase for de novo single-stranded DNA synthesis. Uncontrolled de novo single stranded DNA synthesis, as opposed to controlled, takes advantage of TdT's deoxynucleoside triphosphate (dNTP) 3′ tailing properties on single-stranded DNA to create, for example, homopolymeric adaptor sequences for next-generation sequencing library preparation. In controlled extensions, a reversible deoxynucleoside triphosphate termination technology needs to be employed to prevent uncontrolled addition of dNTPs to the 3′-end of a growing DNA strand. The development of a controlled single-stranded DNA synthesis process through TdT would be invaluable to in situ DNA synthesis for gene assembly or hybridization microarrays as it removes the need for an anhydrous environment and allows the use of various polymers incompatible with organic solvents.

However, TdT has been shown not to efficiently add nucleoside triphosphates containing 3′-O-reversibly terminating moieties for building up a nascent single-stranded DNA chain necessary for a de novo synthesis cycle. A 3′-O-reversible terminating moiety would prevent a terminal transferase such as TdT from catalysing the nucleotide transferase reaction between the 3′-end of a growing DNA strand and the 5′-triphosphate of an incoming nucleoside triphosphate. The inventors have previously discovered certain modified nucleotides can be incorporated using terminal transferases. Modified nucleotides suitable for terminal transferase extension have been disclosed in for example PCT/GB2018/053305. A common reversible terminator is the aminooxy (O—NH₂) group. The aminooxy group is converted to OH by treatment with nitrite. However, the purine nucleobase adenine carries an exocyclic NH₂ group that is also susceptible to reaction with nitrite. Reaction with nitrite leads to deamination, that is, conversion of the exocyclic amine into a carbonyl. This chemical reaction introduces a mutation into the oligonucleotide, for example, deamination of adenine gives rise to the nucleoside inosine, a ‘wobble’ base that can base pair to either T or C, therefore introducing a mutation.

Purines are one of two classes of heterocyclic nitrogenous bases found in both DNA and RNA nucleic acid constructs. Purines found in DNA and RNA nitrogenous bases are adenine (A) and guanine (G). These bases can form hydrogen bonds with their complementary pyrimidines—cytosine (C) in the case of guanine and thymine (T) (DNA) or uracil (U) (RNA) in the case of adenine. Hydrogen bonding is of vital biochemical importance, for instance it is required to form complementary double stranded structures or select the correct tRNAs during protein translation.

Deamination changes the hydrogen bonding pattern of the base and thus alters the base pairing properties of the base. One effect of a deamination mutation is to change the efficiency with which a nucleic acid can hybridise to a target; this effect typically manifests in a decrease in the melting temperature of the duplex. A second effect of a deamination mutation is that a nucleic acid copy (for instance made by a DNA polymerase) will also contain a mutation. A third effect of a deamination mutation is to change the function of the nucleic acid, for example, by changing the amino acid sequence of a resultant peptide/protein should the nucleic acid undergo translation. The protein translated from a mutated nucleic acid would have the wrong sequence, likely fold incorrectly, and ultimately exhibit a loss of or reduction in function. Clearly, mutations are often unacceptable as they affect the properties of the nucleic acid and lead to a change in the encoded information.

### SUMMARY OF THE INVENTION

Disclosed herein a method of reducing the deamination of the adenine base during oligonucleotide synthesis. The method is particularly applicable when nitrite is used to remove an aminooxy terminating moiety from the sugar hydroxyl. The compounds herein are modified in one of three ways. The compounds are either 8-aza modified such that positions 7, 8 and 9 are all N, or positions 2 and/or 8 are modified to contain at least one electron withdrawing group, or a combination thereof.

The modified adenine bases also provide enhanced stability during the conversion to O—NH₂ nucleotides with aminooxy compounds such as methoxylamine.

An aspect of the present invention relates to a compound according to Formula (1a) or (1b):

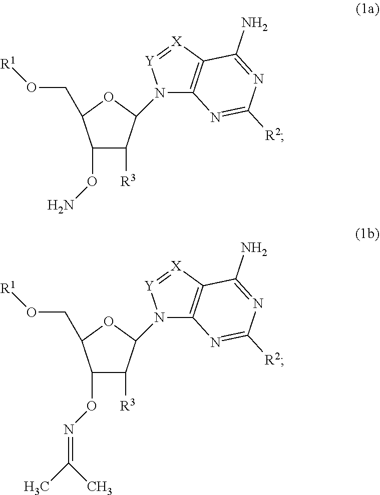

wherein,
R¹ is a phosphate or polyphosphate group or salt thereof, optionally containing one or more sulfur atoms;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.
Where R² is H, either Y is N or X is CR⁸ where R⁸ is an electron withdrawing group (EWG).
Where R² is an electron withdrawing group (EWG), Y can be CH or N and X can be N, CH, CR⁷ or CR⁸.

Known compounds in the art include for example

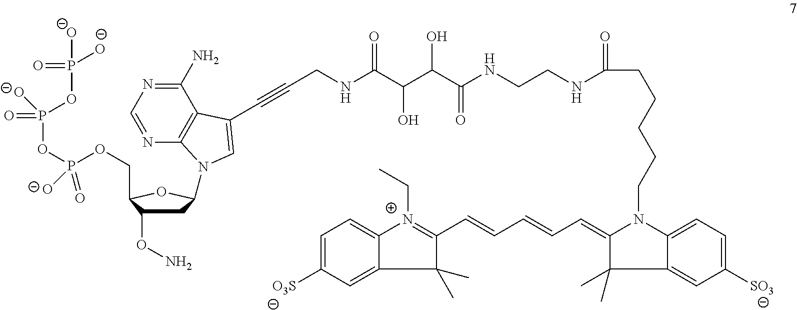

### Hutter et al; Nucleosides Nucleotides Nucleic Acids. 2010 November; 29(11): doi:10.1080/15257770.2010.536191.

Such compounds where R² is H and Y is CH are outside the scope of the claimed invention as they are lacking any electron withdrawing moieties at the 7 position (CR⁸).

A further aspect includes a compound according to Formula (1c) or (1d):

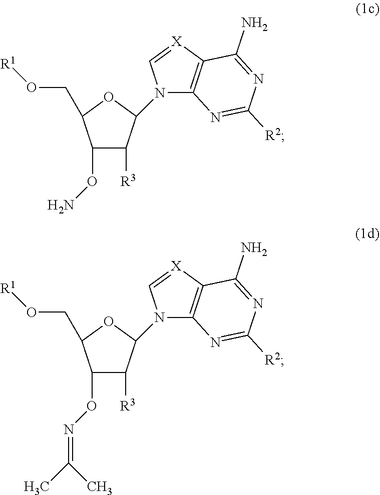

wherein,
R¹ is a phosphate or polyphosphate group or salt thereof, optionally containing one or more sulfur atoms;
R² is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe;
wherein R⁴ and R⁵ are independently selected from H and C₁₋₆ alkyl optionally substituted with OH or halo atoms; and
X is N, CH or CR⁷ where R⁷ is optionally substituted C₁₋₅ alkyl, optionally substituted C₁₋₅ alkenyl or optionally substituted C₁₋₅ alkynyl.

A further aspect includes a compound of Formula (2a) or (2b):

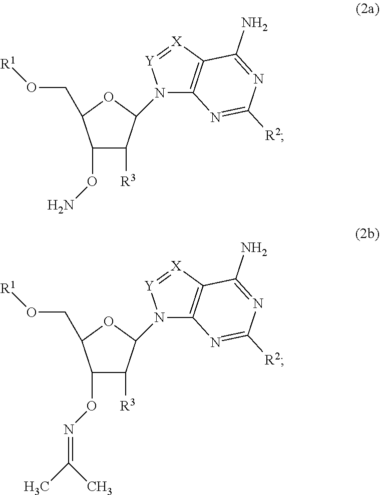

wherein,
R¹ is a phosphate or polyphosphate group or salt thereof, optionally containing one or more sulfur atoms;
R² is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe;
X is N, CH or CR⁷ where R⁷ is optionally substituted C₁₋₅ alkyl, optionally substituted C₁₋₅ alkenyl or optionally substituted C₁₋₅ alkynyl;
Y is CH or N; and
wherein R⁴ and R⁵ are independently selected from H and C₁₋₆ alkyl optionally substituted with OH or halo atoms.

A further aspect includes a compound of Formula (3a) or (3b):

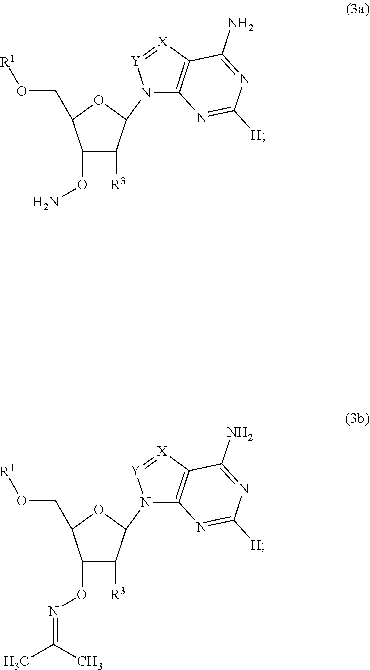

wherein,
R¹ is a phosphate or polyphosphate group or salt thereof, optionally containing one or more sulfur atoms;
R³ is selected from H, OH, F, OCH₃, or OCH₂CH₂OMe; and
X is N or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

A further aspect relates to a method of nucleic acid synthesis comprising reacting a compound herein with an oligonucleotide in the presence of a polymerase or terminal deoxynucleotidyl transferase (TdT) enzyme and treating the extended oligonucleotide with a nitrite salt. The oligonucleotide sequence can be a solid-supported oligonucleotide sequence.

A further aspect relates to a method of synthesizing a compound according to formula (1a), (2a) or (3a) by treating the compounds of Formula (1b), (2b) or (3b) with an aminooxy compound. The aminooxy compound may be hydroxylamine, methoxylamine or ethoxylamine.

A further aspect relates to an oligonucleotide according to Formula (1a) or (1b):

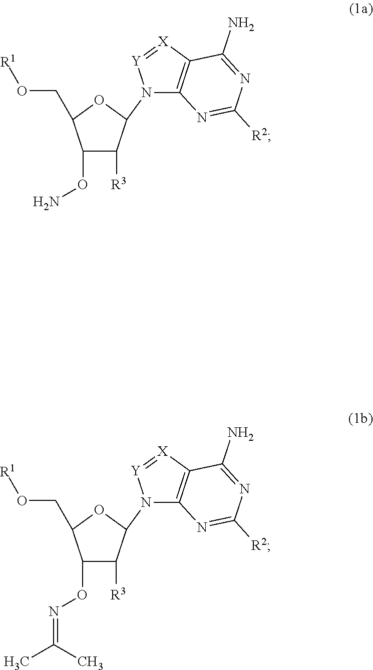

wherein,
R¹ is an oligonucleotide;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

A further aspect of the present invention relates to a kit comprising:
(i) a compound according to any one of Formula (1a) or (1b);
(ii) a terminal deoxynucleotidyl transferase (TdT) enzyme; and optionally
(iii) a nitrite salt.

### DETAILED DESCRIPTION OF THE INVENTION

Disclosed herein is a method of reducing the deamination of the adenine base during oligonucleotide synthesis. The method is particularly applicable when nitrite is used to convert an aminooxy terminating moiety on the sugar to a hydroxyl. Electron withdrawing groups (EWG) in the 2-, 7- or 8-positions of adenine can dramatically reduce nitrosative deamination. These EWG in the 2-, 7- or 8-positions can increase the stability of adenine molecules relative to the parent compound. In particular, chloro and fluoro substituents at the 2- and 7 position decrease the rate of nitrite-mediated deamination by up to an order of magnitude. There is a significant industrial applicability because deamination changes the identity and hydrogen bonding pattern of the base, i.e. deamination introduces mutations into the product. Mutations are undesirable as they lead to change in sequence of the DNA, and thus affect the biophysical properties, biochemical properties, and information encoding properties of the DNA.

2-position modified adenine nucleotides are of value to enzymatic DNA synthesis when using 3′-O-aminooxy reversible terminators or the precursors thereof. While adenine present in a synthesised strand will undergo a level of nitrite-mediated deamination that introduces mutations, 2-position electron withdrawing modified adenines are more robust and thus yield a higher quality product.

The 3′-O-aminooxy reversible terminator precursors may include where the aminooxy is protected as an oxime, for example N═C(CH₃)₂. The oxime can be transformed into aminooxy as part of the unblocking process. The modified adenine bases provide enhanced stability during the conversion of O—N═C(CH₃)₂ to O—NH₂ nucleotides with aminooxy compounds such as methoxylamine.

The compounds described either have an electron withdrawing group at the 2 and/or 7 positions or have a 8-aza modification.

An aspect of the present invention relates to a compound according to Formula (1a) or (1b):

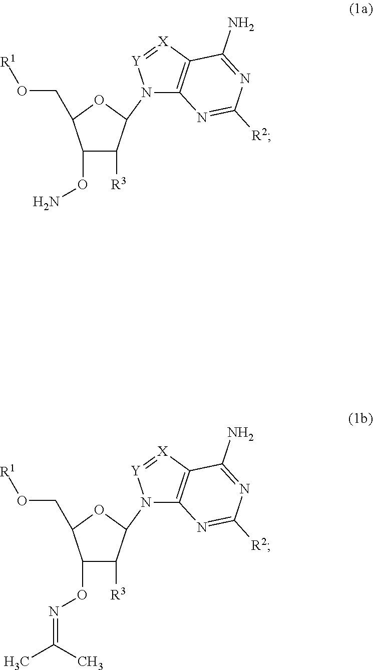

wherein,
R¹ is a phosphate or polyphosphate group or salt thereof, optionally containing one or more sulfur atoms;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

Also described is a compound according to Formula (1c) or (1d):

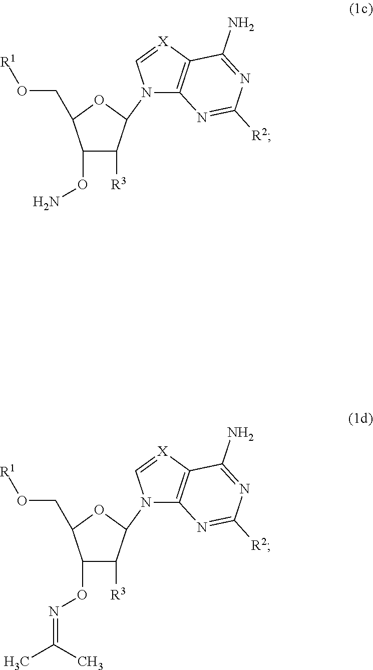

wherein,
R¹ is a phosphate or polyphosphate group or salt thereof, optionally containing one or more sulfur atoms;
R² is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe;
wherein R⁴ and R⁵ are independently selected from H and C₁₋₆ alkyl optionally substituted with OH or halo atoms; and
X is N, CH or CR⁷ where R⁷ is optionally substituted C₁₋₅ alkyl, optionally substituted C₁₋₅ alkenyl or optionally substituted C₁₋₅ alkynyl.

An aspect of the invention involves converting compounds of Formula (1b) to compounds of Formula (1a). The conversion may be performed using aminooxy compounds. The conversion may be performed using methoxylamine. Disclosed is a method of synthesizing a compound according to formula (1a):

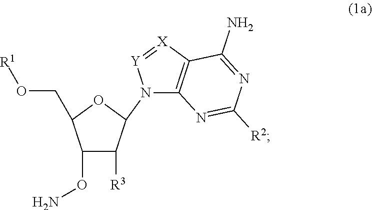

wherein,
R¹ is a phosphate or polyphosphate group or salt thereof, optionally containing one or more sulfur atoms;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms; using a compound according to Formula (1b):

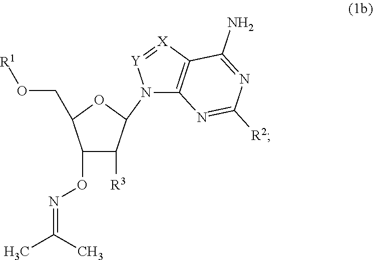

wherein,
R¹ is a phosphate or polyphosphate group or salt thereof, optionally containing one or more sulfur atoms;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

Disclosed is a method of synthesizing a compound according to formula (1a); comprising taking a compound according to Formula (1b) and treating the compounds of Formula (1b) with an aminooxy compound. The aminoxy compound may be hydroxylamine, methoxylamine or ethoxylamine. R¹ can be a phosphate or polyphosphate group. The phosphate groups can be protonated or in salt form. The phosphates can be entirely oxygen, or can contain one or more sulfur atoms. R¹ can be a phosphate group. R¹ can be a polyphosphate group. R¹ can also be a phosphate or polyphosphate group selected from —(PO₃)⁻ₓ(PO₂S)⁻_{y}(PO₃)⁻_{z} where x, y and z are independently 0-5 and x+y+z is 1-5. R¹ can also be a phosphate or polyphosphate group having one or more sulfur atoms. R¹ can be a phosphate group having one or more sulfur atoms. R¹ can be a polyphosphate group having one or more sulfur atoms. The sulfur atom can be in any position on any on the phosphate groups. R¹ can further be a monophosphate, diphosphate, triphosphate, tetraphosphate, pentaphosphate, or (alpha-thio)triphosphate group. R¹ can be a monophosphate group. R¹ can be a diphosphate group. R¹ can be a tetraphosphate group. R¹ can be a pentaphosphate group. R¹ can be an (alpha-thio)triphosphate group. R¹ can be a triphosphate group. R¹ can be an oligonucleotide.

R² is an electron withdrawing group (EWG). R² can be an electron withdrawing group (EWG) that can be selected from the group consisting of halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵. R² can be a halo group. R² can be selected from F, Cl, Br or I. R² can be F or Cl. R² can be a nitrile group. R² can be a nitro group. R² can be a nitrosyl group. R² can be halo, halomethyl, dihalomethyl or trihalomethyl. R² can be a halomethyl group. R² can be a dihalomethyl group. R² can be a trihalomethyl group. R² can be a C≡CR⁴ group. R² can be a SOR⁴; SO₂R⁴ or SO₃R⁴ group. R² can be an electron withdrawing group (EWG) consisting of a COR⁴ group such as an aldehyde or ketone. R² can be an electron withdrawing group (EWG) consisting of a CO₂R⁴ group such as an acid or ester. R² can be an electron withdrawing group (EWG) consisting of an amide CONR⁴R⁵ group.

R⁴ and R⁵ can be independently selected from H and C₁₋₆ alkyl optionally substituted with OH or halo atoms. R⁴ and R⁵ can be independently selected from H and C₁₋₆ alkyl optionally substituted with OH or 1-6 halo atoms. R⁴ can be H. R⁴ can be C₁₋₆ alkyl optionally substituted with OH or halo atoms, wherein the halo atoms can be selected from F, Cl, Br or I. R⁴ can be C₁₋₆ alkyl optionally substituted with OH or 1-6 halo atoms, wherein the halo atoms can be selected from F, Cl, Br or I. R⁴ can be CH₃. R⁴ can be CH₂OH. R⁴ can be CH₂CH₂OH.

R⁵ can be H. R⁵ can be C₁₋₆ alkyl optionally substituted with OH or halo atoms. R⁵ can be C₁₋₆ alkyl optionally substituted with OH or 1-6 halo atoms. R⁵ can be C₁₋₆ alkyl optionally substituted with OH or halo atoms, wherein the halo atoms can be selected from F, Cl, Br or I. R⁵ can be C₁₋₆ alkyl optionally substituted with OH or 1-6 halo atoms, wherein the halo atoms can be selected from F, Cl, Br or I. R⁵ can be CH₃.

One embodiment of the present invention relates to a compound according to Formula (1a) or (1b) wherein R² can be selected from the group consisting of fluoro, chloro or CF₃. R² can be fluoro. R² can be chloro. R² can be CF₃.

R³ can be selected from H, OH, F, OCH₃ or OCH₂CH₂OMe. R³ can be OH. R³ can be F. R³ can be OCH₃. R³ can be OCH₂CH₂OMe. Preferably, R³ can be H.

X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵. Where X is N, Y must be also be N, or the 2-position must be an electron withdrawing group (i.e. R₂ is not H). Where R2 is an electron withdrawing group, X can be any of N, CH or CR⁷.

Optionally both R⁸ and R² can independently be different electron withdrawing groups.

Y is CH or N. Where Y is CH, either R² and/or R⁸ must be an EWG. Where Y is N, X can be N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵. Where Y is N, R² can be H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;

There the compound must satisfy that either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² can be an electron withdrawing group (EWG) that can be selected from the group consisting of halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵. R² can be an electron withdrawing group (EWG) consisting of a halo group. R² can be an electron withdrawing group (EWG) consisting of a halo group which can be selected from F, Cl, Br or I. R² can be F or Cl. R² can be an electron withdrawing group (EWG) consisting of a nitrile group. R² can be an electron withdrawing group (EWG) consisting of a halomethyl group. R² can be an electron withdrawing group (EWG) consisting of a dihalomethyl group. R² can be an electron withdrawing group (EWG) consisting of a trihalomethyl group. R² can be an electron withdrawing group (EWG) consisting of a C≡CR⁴ group. R² can be an electron withdrawing group (EWG) consisting of a SOR⁴ group. R² can be an electron withdrawing group (EWG) consisting of a SO₂R⁴ group. R² can be an electron withdrawing group (EWG) consisting of a COR⁴ group. R² can be an electron withdrawing group (EWG) consisting of a CO₂R⁴ group. R² can be an electron withdrawing group (EWG) consisting of a CONR⁴R⁵ group.
R⁸ can be an electron withdrawing group (EWG) that can be selected from the group consisting of halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵. R⁸ can be an electron withdrawing group (EWG) consisting of a halo group. R⁸ can be an electron withdrawing group (EWG) consisting of a halo group which can be selected from F, Cl, Br or I. R² can be F or Cl. R⁸ can be an electron withdrawing group (EWG) consisting of a nitrile group. R⁸ can be an electron withdrawing group (EWG) consisting of a halomethyl group. R⁸ can be an electron withdrawing group (EWG) consisting of a dihalomethyl group. R⁸ can be an electron withdrawing group (EWG) consisting of a trihalomethyl group. R⁸ can be an electron withdrawing group (EWG) consisting of a C≡CR⁴ group. R⁸ can be an electron withdrawing group (EWG) consisting of a SOR⁴ group. R⁸ can be an electron withdrawing group (EWG) consisting of a SO₂R⁴ group. R⁸ can be an electron withdrawing group (EWG) consisting of a COR⁴ group. R⁸ can be an electron withdrawing group (EWG) consisting of a CO₂R⁴ group. R⁸ can be an electron withdrawing group (EWG) consisting of a CONR⁴R⁵ group.

The compounds of Formula (1a) or (1b) can be selected from the group consisting of:

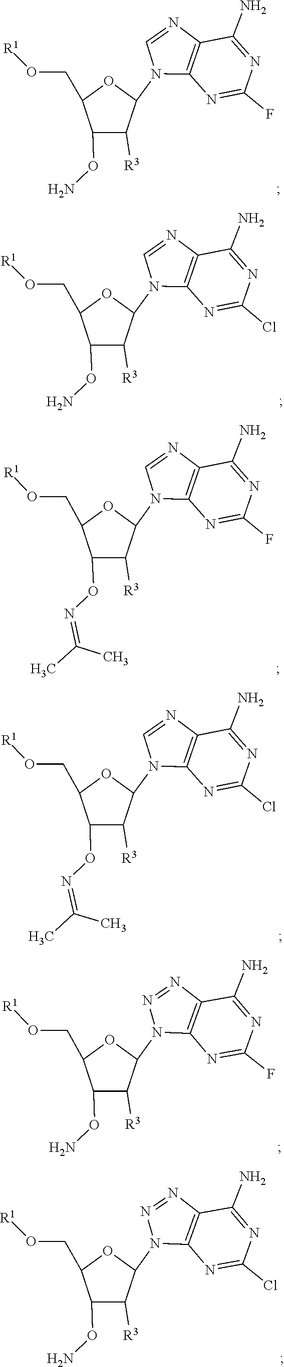

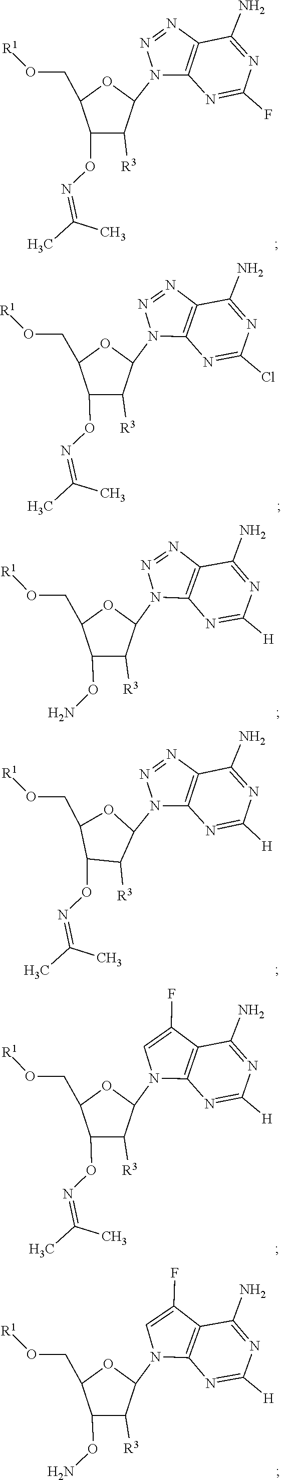

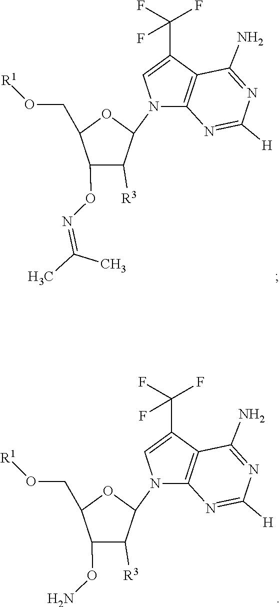

wherein, R¹ is a phosphate or polyphosphate group or salt thereof, optionally containing one or more sulfur atoms or R¹ is an oligonucleotide.

The compounds of Formula (1a) or (1b) can also be selected from the group consisting of:

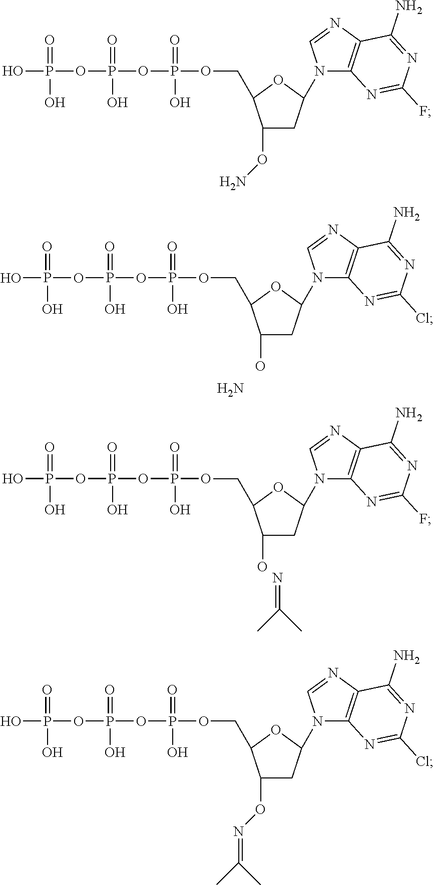

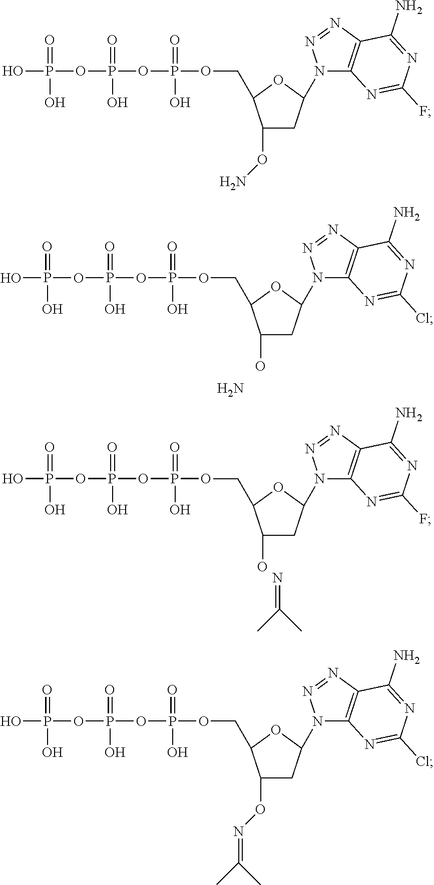

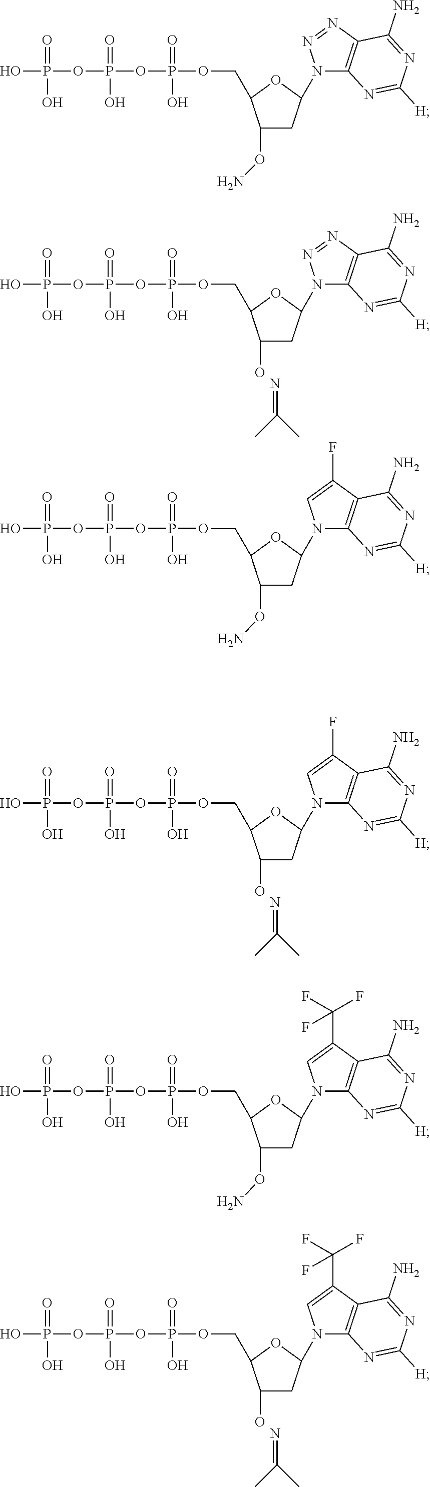

or a salt thereof.

Included herein is a method of nucleic acid synthesis comprising reacting a compound of Formula (1a) or (1b) with an oligonucleotide in the presence of a polymerase or terminal deoxynucleotidyl transferase (TdT) enzyme and treating the extended oligonucleotide with a nitrite salt.

The terminal transferase or modified terminal transferase can be any enzyme capable of template independent strand extension. The modified terminal deoxynucleotidyl transferase (TdT) enzyme can comprise amino acid modifications when compared to a wild type sequence or a truncated version thereof. The terminal transferase can be the homologous amino acid sequence of a terminal deoxynucleotidyl transferase (TdT) enzyme in any species or the homologous amino acid sequence of Polμ, Polβ, PolΞ, and Polθ of any species or the homologous amino acid sequence of X family polymerases of any species.

Homologous refers to protein sequences between two or more proteins that possess a common evolutionary origin, including proteins from superfamilies in the same species of organism as well as homologous proteins from different species. Such proteins (and their encoding nucleic acids) have sequence homology, as reflected by their sequence similarity, whether in terms of percent identity or by the presence of specific residues or motifs and conserved positions. A variety of protein (and their encoding nucleic acid) sequence alignment tools may be used to determine sequence homology. For example, the Clustal Omega multiple sequence alignment program provided by the European Molecular Biology Laboratory (EMBL) can be used to determine sequence homology or homologous regions.

A further embodiment of the present invention relates to the oligonucleotide sequence comprising a solid-supported oligonucleotide sequence. The oligonucleotide sequence comprises 2 or more nucleotides. The oligonucleotide sequence can be between 10 and 500 nucleotides, such as between 20 and 200 nucleotides, in particular between 20 and 50 nucleotides long.

A further embodiment of the present invention relates to a method further comprising a reaction step with a nitrite salt. Preferably, the nitrate salt is sodium nitrite.

A further aspect of the present invention relates to a kit comprising:
(i) a compound according to any one of Formula (1a) or (1b);
(ii) a polymerase or terminal deoxynucleotidyl transferase (TdT) enzyme; and optionally
(iii) a nitrite salt.

A further aspect of the present invention relates to an oligonucleotide according to Formula (1a) or (1b).

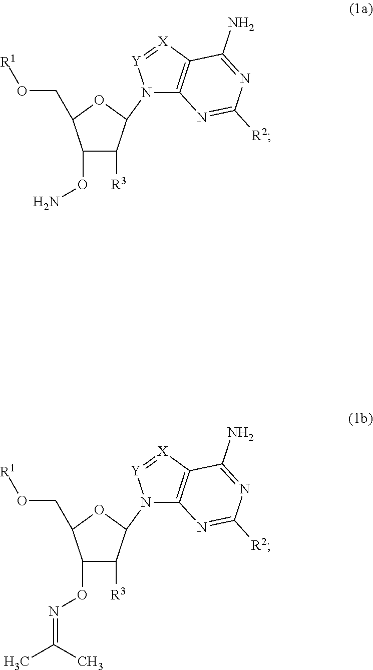

wherein,
R¹ is an oligonucleotide;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

A further embodiment of the present invention relates to an oligonucleotide according to Formula (2a) or (2b) wherein R¹ can be an oligonucleotide. The phosphates in R¹ can contain one or more sulfur atoms.

A further embodiment of the present invention relates to a compound according to Formula (1a) or (1b) or (2a) or (2b) or (3a) or (3b) wherein R² can be an electron withdrawing group (EWG). R² and/or R⁸ can be an electron withdrawing group (EWG) that can be selected from the group consisting of halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵. R² and/or R⁸ can be an electron withdrawing group (EWG) consisting of a halo group. R² and/or R⁸ can be an electron withdrawing group (EWG) consisting of a halo group which can be selected from F, Cl, Br or I. R² and/or R⁸ can be F or Cl. R² and/or R⁸ can be an electron withdrawing group (EWG) consisting of a nitrile group. R² and/or R⁸ can be an electron withdrawing group (EWG) consisting of a halomethyl group. R² and/or R⁸ can be an electron withdrawing group (EWG) consisting of a dihalomethyl group. R² and/or R⁸ can be an electron withdrawing group (EWG) consisting of a trihalomethyl group. R² and/or R⁸ can be an electron withdrawing group (EWG) consisting of a C≡CR⁴ group. R² and/or R⁸ can be an electron withdrawing group (EWG) consisting of a SOR⁴ group. R² and/or R⁸ can be an electron withdrawing group (EWG) consisting of a SO₂R⁴ group. R² and/or R⁸ can be an electron withdrawing group (EWG) consisting of a COR⁴ group. R² and/or R⁸ can be an electron withdrawing group (EWG) consisting of a CO₂R⁴ group. R² and/or R⁸ can be an electron withdrawing group (EWG) consisting of a CONR⁴R⁵ group.

A further embodiment of the present invention relates to an oligonucleotide according to Formula (1a) or (1b) wherein R³ can be selected from H, OH, F, OCH₃, or OCH₂CH₂OMe. R³ can be OH. R³ can be F. R³ can be OCH₃. R³ can be OCH₂CH₂OMe. Preferably, R³ can be H.

A further embodiment of the present invention relates to a compound according to Formula (1a) or (1b) wherein R⁴ can be independently selected from H and C₁₋₆ alkyl optionally substituted with OH or halo atoms. R⁴ can be H. R⁴ can be C₁₋₆ alkyl optionally substituted with OH or halo atoms, wherein the halo atoms can be selected from F, Cl, Br or I. R⁴ can be C₁₋₆ alkyl optionally substituted with OH or 1-6 halo atoms, wherein the halo atoms can be selected from F, Cl, Br or I.

A further embodiment of the present invention relates to a compound according to Formula (1a) or (1b) wherein R⁵ can be independently selected from H and C₁₋₆ alkyl optionally substituted with OH or halo atoms. R⁵ can be H. R⁵ can be C₁₋₆ alkyl optionally substituted with OH or halo atoms, wherein the halo atoms can be selected from F, Cl, Br or I. R⁵ can be C₁₋₆ alkyl optionally substituted with OH or 1-6 halo atoms, wherein the halo atoms can be selected from F, Cl, Br or I.

Described herein is a process of nucleic acid synthesis using the compounds described herein. The process uses a nucleic acid polymerase, which may be a template independent polymerase or a template dependent polymerase to add a single nucleotide to one or more nucleic acid strands. The strands may be immobilised on a solid support. The process involves cleaving the 3′-aminooxy group and adding a further nucleotide, the base of which may or may not be a purine as described herein.

Disclosed is a method of nucleic acid synthesis comprising reacting a compound described herein with an oligonucleotide in the presence of a polymerase or terminal deoxynucleotidyl transferase (TdT) enzyme and treating the extended oligonucleotide with a nitrite salt.

In the methods of nucleic acid synthesis described herein the oligonucleotide sequence may be a solid-supported oligonucleotide sequence.

In the methods of nucleic acid synthesis described herein the nitrite salt may be sodium nitrite.

Disclosed is a method of nucleic acid synthesis comprising:
(a) providing an initiator sequence;
(b) adding extension reagents comprising a polymerase or terminal deoxynucleotidyl transferase (TdT) and a compounds according to Formula (1a) or (1b):
(c) removal of the extension reagents;
(d) optionally transforming the N=C(CH₃)₂ if present to NH₂;
(e) converting the 3′-O—NH₂ group on the extended nucleic acid polymer to a 3′-OH group;
(f) adding extension reagents comprising a 3′-O—NH₂ or 3′-O—N═C(CH₃)₂ blocked nucleoside triphosphate and a polymerase or terminal deoxynucleotidyl transferase (TdT) to said initiator sequence to add a further single nucleotide to the initiator sequence.

The nucleic acids synthesised can be any sequence. One or more, possibly all, of the adenine bases will have the electron withdrawing group at the 2-position. A population of different sequences can be synthesised in parallel.

Where the cytosine or guanine heterocyclic bases have exocyclic NH₂ groups, these groups can optionally be masked by an orthogonal masking agent. The amine masked nitrogenous heterocycles may be N4-amine masked cytidine and N2-amine masked guanine. The masking may be for example an azido (N₃) group. Example for suitable masking groups include azide (−N₃), benzoylamine (N-benzoyl or —NHCOPh), N-methyl (—NHMe), isobutyrylamine, dimethylformamidylamine, 9-fluorenylmethyl carbamate, t-butyl carbamate, benzyl carbamate, acetamide (N-acetyl or —NHCOMe), trifluoroacetamide, pthlamide, benzylamine (N-benzyl or —NH—CH₂-phenyl), triphenylmethylamine, benxylideneamine, tosylamide, isothiocyanate, N-allyl (such as N-dimethylallyl (—NHCH₂—CH═CH₂)) and N-anisoyl (—NHCOPh-OMe), such as azide (—N₃), N-acetyl (—NHCOMe), N-benzyl (—NH-CH₂-phenyl), N-anisoyl (—NHCOPh-OMe), N-methyl, (—NHMe), N-benzoyl (—NHCOPh), N-dimethylallyl (—NHCH₂—CH═CH₂).

References herein to an “amine masking group” refer to any chemical group which is capable of generating or “unmasking” an amine group which is involved in hydrogen bond base-pairing with a complementary base. Most typically the unmasking will follow a chemical reaction, most suitably a simple, single step chemical reaction. The amine masking group will generally be orthogonal to the 3′-O—NH₂ blocking group in order to allow selective removal.

In the nucleic acids synthesised, the bases can be selected from: T or modified T such as for example ‘super-T’; C or a modified C such as for example a C having an electron withdrawing group at the 5 position, as described herein; A or a modified A such as for example an N6-amine masked adenine; and G or a modified G such as for example an N2-amine masked guanine. The amino masking group prevents de-amination caused by the nitrite exposure needed to remove the O—NH₂ at the 3′-position of the sugar.

The T nucleotides can be selected from

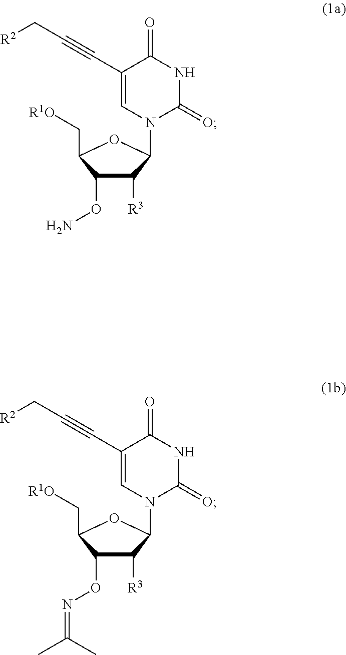

wherein, R¹ is a phosphate or polyphosphate group or salt thereof, optionally containing one or more sulfur atoms;
R² is H, halo, OH, NH₂, COOH, COH, C₁₋₃ alkoxy, C₁₋₃ alkyl optionally substituted with OH, NH₂ or halo atoms; and
R³ is selected from H, OH, F, OCH₃ or OCH₂CH₂OMe.

The T nucleotides can be

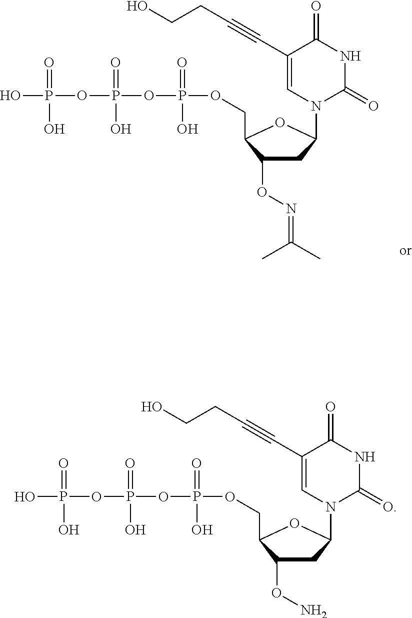 [SMILES: CC(C)=NO[C@@H]1C[C@H](n2cc(C#CCCO)c(=O)[nH]c2=O)O[C@@H]1COP(=O)(O)OP(=O)(O)OP(=O)(O)O ; NO[C@@H]1C[C@H](n2cc(C#CCCO)c(=O)[nH]c2=O)O[C@@H]1COP(=O)(O)OP(=O)(O)OP(=O)(O)O]

or a salt thereof.

The guanine compounds may be selected from:

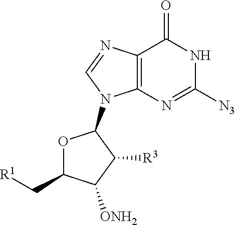

where R¹ and R³ are as defined herein.

The term ‘azide’ or ‘azido’ used herein refers to an —N₃, or more specifically, an —N═N⁺═N⁻ group. It will also be appreciated that azide extends to the presence of a tetrazolyl moiety. The “azide-tetrazole” equilibrium is well known to the skilled person from Lakshman et al (2010) J. Org. Chem. 75, 2461-2473. Thus, references herein to azide extend equally to tetrazole as illustrated below when applied to the R³ groups defined herein:

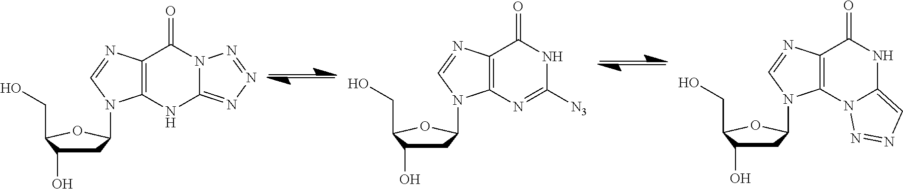

This embodiment has the advantage of reversibly masking the —NH₂ group. While blocked in the —N₃ state, the base (B) is impervious to deamination (e.g., deamination in the presence of sodium nitrite). The base (B) in the N-blocked form is incapable of forming secondary structures via base pairing. Thus, even blocking a subset of the free amino groups in the nucleic acid polymer improves the availability of the 3′-end for further extension. The canonical guanine can be respectively recovered from 2-azido guanine by exposure to a reducing agent (e.g., TCEP). Thus, the —N₃ group serves as an effective protecting group against deamination, especially in the presence of sodium nitrite.

Alternatively the G bases may be modified at the 7 or 8 positions in a similar manner to the modifications described herein. The G nucleoside may be of formula:

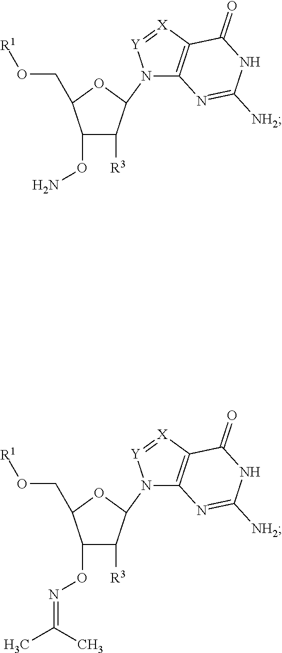

wherein,
R¹ is a phosphate or polyphosphate group or salt thereof, optionally containing one or more sulfur atoms;
R³ is selected from H, OH, F, OCH₃, or OCH₂CH₂OMe; and either
X is N and Y is N, or X is CR² and Y is CH or N, where R² is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; wherein R⁴ and R⁵ are independently selected from H and C₁₋₆ alkyl optionally substituted with OH or halo atoms.

It will be appreciated that the compounds of the invention may be readily applied to methods of enzymatic nucleic acid synthesis which are well known to the person skilled in the art. Non-limiting methods of nucleic acid synthesis may be found in WO 2016/128731, WO 2016/139477, WO 2017/009663, GB 1613185.6 and GB 1714827.1, the contents of each of which are herein incorporated by reference.

Enzymatic nucleic acid synthesis is defined as any process in which a nucleotide is added to a nucleic acid strand through enzymatic catalysis in the presence or absence of a template. For example, a method of enzymatic nucleic acid synthesis could include non-templated de novo nucleic acid synthesis utilizing a PolX family polymerase, such as terminal deoxynucleotidyl transferase, and reversibly terminated 2′-deoxynucleoside 5′-triphosphates or ribonucleoside 5′-triphosphate. Another method of enzymatic nucleic acid synthesis could include templated nucleic acid synthesis, including sequencing-by-synthesis. Reversibly terminated enzymatic nucleic acid synthesis is defined as any process in which a reversibly terminated nucleotide is added to a nucleic acid strand through enzymatic catalysis in the presence or absence of a template. Thus, in one embodiment, the method of enzymatic nucleic acid synthesis is selected from a method of reversibly terminated enzymatic nucleic acid synthesis and a method of templated and non-templated de novo enzymatic nucleic acid synthesis.

References herein to ‘nucleoside triphosphates’ refer to a molecule containing a nucleoside (i.e. a base attached to a deoxyribose or ribose sugar molecule) bound to three phosphate groups. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates, such as naturally occurring modified nucleosides and artificial/modified/non-naturally occurring nucleosides.

Therefore, references herein to ‘3′-blocked nucleoside triphosphates’ refer to nucleoside triphosphates (e.g., dATP, dGTP, dCTP or dTTP) which have an additional group on the 3′-end which prevents further addition of nucleotides, i.e., by replacing the 3′-OH group with a protecting group. Herein the protecting group is NH₂ or a protected version thereof.

References herein to a ‘DNA initiator sequence’ refer to a small sequence of DNA which the 3′-blocked nucleoside triphosphate can be attached to, i.e., DNA will be synthesised from the 3′-end of the DNA initiator sequence.

In one embodiment, the initiator sequence is between 5 and 100 nucleotides long, such as between 10 and 60 nucleotides long, in particular between 20 and 50 nucleotides long. The ideal length of initiator may be informed by the immobilisation state (i.e. in solution or immobilised), the immobilisation chemistry, the initiator base sequence, and other parameters.

In one embodiment, the initiator sequence is single-stranded. In an alternative embodiment, the initiator sequence is double-stranded. In a further embodiment, the initiator sequence has double-stranded and single-stranded portions. It will be understood by persons skilled in the art that a 3′-overhang (i.e., a free 3′-end) allows for efficient addition.

In one embodiment, the initiator sequence is immobilised on a solid support. This allows the enzyme and the cleaving agent to be removed without washing away the synthesised nucleic acid. The initiator sequence may be attached to a solid support stable under aqueous conditions so that the method can be easily performed via a flow setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In one embodiment, the initiator sequence contains a base or base sequence recognisable by an enzyme. A base recognised by an enzyme, such as a glycosylase, may be removed to generate an abasic site which may be cleaved by chemical or enzymatic means. An example of such a glycosylase system includes the presence of a uracil base in the initiator sequence, which may be excised with uracil DNA glycosylase (UDG) to leave an abasic site which may be cleaved with, for example, basic solutions, organic amines, or an endonuclease (such as endonuclease VIII), to release a nucleic acid bearing a 5′-phosphate into solution. A base sequence may be recognised and cleaved by a restriction enzyme.

In a further embodiment, the initiator sequence is immobilised on a solid support via an orthogonal chemically-cleavable linker, such as a disulfide, allyl, or azide-masked hemiaminal ether linker. Therefore, in one embodiment, where an azido N-masking group is not present, the method additionally comprises extracting the resultant nucleic acid by cleaving the chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker.

In one embodiment, the resultant nucleic acid is extracted and amplified by polymerase chain reaction (PCR) using the nucleic acid bound to the solid support as a template. The initiator sequence could therefore contain an appropriate forward primer sequence and an appropriate reverse primer could be synthesised or incorporated via ligation.

In one embodiment, the terminal deoxynucleotidyl transferase (TdT) of the invention is added in the presence of an extension solution comprising one or more buffers (e.g., Tris or cacodylate), one or more salts (e.g., Na⁺, K⁺, Mg²⁺, Mn²⁺, Cu²⁺, Zn²⁺, CO²⁺, etc. all with appropriate counterions, such as Cl) and inorganic pyrophosphatase (e.g., the Saccharomyces cerevisiae homolog). It will be understood that the choice of buffers and salts depends on the optimal enzyme activity and stability. The use of an inorganic pyrophosphatase helps to reduce the build-up of pyrophosphate due to nucleoside triphosphate hydrolysis by TdT. Therefore, the use of an inorganic pyrophosphatase has the advantage of reducing the rate of (1) backwards reaction and (2) TdT strand dismutation.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

The compounds of the invention can be used on a device for nucleic acid synthesis. In one embodiment of the invention there is a solid support in the form of for example a planar array and further a plurality of beads onto which a plurality of immobilized initiation oligonucleotide sequences are attached. The beads may be porous and a portion of the, optionally porous, beads are selected as anchors and unselected beads are exposed to harvest solution to cleave them from their solid support to release the oligonucleotide sequences into solution. Thus the term solid support can refer to an array having a plurality of beads which may or may not be immobilised. The oligonucleotides may be attached to, or removed from beads whilst on the array. Thus the immobilised oligonucleotide may be attached to a bead, which remains in a fixed position on the array whilst other beads in other locations are subject to cleavage conditions to detach the oligonucleotides from the beads (the beads may or may not be immobilised).

The solid support can take the form of a digital microfluidic device. Digital microfluidic devices consist of a plurality of electrodes arranged on a surface. A dielectric layer (e.g., aluminum oxide) is deposited over the electrodes followed by a hydrophobic coating (e.g., perfluorinated hydrocarbon polymer) atop the dielectric layer. The electrodes may be hardwired or formed from an active matrix thin film transistor (AM-TFT).

The solid support can take the form of a digital microfluidic device. Digital microfluidic devices consist of a plurality of electrodes arranged on a surface. These electrodes can be addressed in a passive manner or by active matrix methods. Passive addressing is a direct address where actuation signals are directly applied on individual electrode (for example by means of a hard-wired connection to that electrode in a single layer or multilayer fashion such as a printed circuit board, PCB). However, a limitation of direct drive methods is the inability to process large numbers of droplets due to difficulties in addressing large numbers of direct drive electrodes. In active matrix addressing, MxN electrodes can be controlled by M+N pins, significantly reducing the number of control pins. However, the resolution of the electrodes (size of electrodes as compared to the size of droplets) limits the scope of droplet operations. Active matrix thin film transistor (AM-TFT) technology enables the control of large numbers of droplets by replacing patterned electrodes with a thin film transistor array, each of which is individually addressable. The increased resolution (small size of pixels on the thin film transistor array) also increases the scope of droplet operations. An AM-TFT digital microfluidic device comprises a dielectric layer (e.g., aluminum oxide) deposited over the electrode layer on the thin-film transistor layer followed by a hydrophobic coating (e.g., perfluorinated hydrocarbon polymer) atop the dielectric layer.

Depending on applied voltage to a subset of the plurality of electrodes arranged on the aforementioned surface, aqueous droplets may be actuated across the surface immersed in oil, air, or another fluid. Enzymatic oligonucleotide synthesis can be deployed on a digital microfluidic device in several ways. An initiator oligonucleotide can be immobilized via the 5′-end on super paramagnetic beads or directly to the hydrophobic surface of the digital microfluidic device. A plurality of distinct positions containing immobilized initiator oligonucleotides on the digital microfluidic device may be present (henceforth named synthesis zones). Solutions required for enzymatic oligonucleotide synthesis are then dispensed from multiple reservoirs onto the device. Briefly, an addition solution containing the components necessary for the TdT-mediated incorporation of reversibly terminated nucleoside 5′-triphosphates onto immobilized initiator oligonucleotides can be dispensed from a reservoir in droplets and actuated to the aforementioned positions containing immobilized initiator oligonucleotides. During this stage, each reservoir (and thus each droplet containing addition solution) can contain a distinct nitrogenous base reversibly terminated nucleoside 5′-triphosphate identity or a mixture thereof in order to control the sequence synthesized on aforementioned positions containing immobilized initiator oligonucleotides.

Alternatively the method can be implemented on continuous flow microfluidic devices. One such device consists of a surface with a plurality of microwells each containing a bead. On said bead, an oligonucleotide initiator can be immobilized. In addition to each microwell containing a bead with immobilized initiator, each microwell can contain an electrode to perform electrochemistry. Another implementation of continuous flow microfluidics consists of a fritted column containing beads or resin on which initiator sequences are immobilized. Addition, wash, and deblocking solutions may be sequentially flowed through the column in a process of DNA synthesis.

In all examples of nucleic acid synthesis, the use of the modified adenine bases having the electron withdrawing groups improves the quality of the synthesised strands due to lowering the level of deamination.

### EXAMPLES

### Stability Studies on 2-halogenated 2′-deoxyadenosines

Analytical LC/MS was performed on the Agilent 1100 Series system. Nucleosides were analysed using gradient of acetonitrile in 20 mM NH₄OAc, pH 4.5. The 1 M stock solution of NH₄OAc, pH 4.5 was prepared by dissolving NH₄OAc (g) in H₂O and adjusted to pH 4.5 with acetic acid. This was further diluted with water to the concentrations required for the chromatography.

### LC/MS Method for Analysis of Polar Compounds

Column: Ascentis Express C18 15×4.6 mm, 5 μm; Column temperature 30° C., Flow rate 1 mL/min, UV detection 254 nm.

| 20 mM NH₄OAc pH 4.5 | A |
|---|---|
| Acetonitrile | B |
| Time (min) | % B |
| 0 | 1 |
| 1 | 1 |
| 10 | 50 |
| 12 | 50 |
| 13 | 1 |

1. Stability of 2-chloro-2′-deoxyadenosine in Oxime Removal Solution (1×ORS ) buffer

Stock solution of ORS buffer (2×) was prepared from methoxyamine hydrochloride (60 mg, 0.71 mmol), water (MilliQ) (200 uL), pH 5.5 sodium acetate (200 uL) and 10 M sodium hydroxide (53.4 uL) and water (1.4 mL). 2-chloro-2′-deoxyadenosine (1 mg) was dissolved in milliQ water (0.5 mL) and 2×ORS (0.5 mL) was added. The resultant solution was incubated at 37 deg C. and the stability was monitored at intervals using LC/MS. Incubation of 2-chloro-2′-deoxyadenosine in 1×ORS buffer for 24 h at 37 deg C. did not lead to any products.
LC/MS analysis of commercial 2-chloro-2′-deoxyadenosine (UV peak at 5.922 min showed MS spectra (m/z 286.1 [M+1]+; m/z 284.1 [M−1]−) which can be assigned to structure of 2-chloro-2′-deoxyadenosine, Mcalc=285.69) (FIG. 1).
Stability of 2-chloro-2′-deoxyadenosine incubated in 1×ORS buffer for 24 h (UV peak at 5.907 min showed MS spectrum (m/z 286.2 [M+1]+) which can be assigned to structure of 2-chloro-2′-deoxyadenosine) (FIG. 1).
   2. Stability of 2-fluoro-2′-deoxyadenosine in ORS Buffer

2-fluoro-2′-deoxyadenosine (1 mg) was dissolved in milliQ water (0.5 mL) and 2×ORS (0.5 mL) was added. The resultant solution was incubated at 37 deg C. and the stability was monitored at intervals using LC/MS. Incubation of 2-fluoro-2′-deoxyadenosine in 1×ORS buffer for 24 h at 37 deg C. did not lead to any products.
LC/MS analysis of commercial 2-fluoro-2′-deoxyadenosine (UV peak at 5.484 min showed

MS spectra (m/z 270.2 [M+1]+; m/z 268.1 [M−1]−) which can be assigned to structure of 2-fluoro-2′-deoxyadenosine, Mcalc=269.24) (FIG. 2).
Stability of 2-fluoro-2′-deoxyadenosine incubated in 1×ORS buffer for 24 h (UV peak at 5.453 min showed MS spectrum (m/z 270.1 [M+1]+) which can be assigned to structure of 2-fluoro-2′-deoxyadenosine, Mcalc=269.24) (FIG. 2).
3. Stability of 2-chloro-2′-deoxyadenosine, 2-fluoro-2′-deoxyadenosine and 2′-deoxyadenosine in NDS Buffer

2 M sodium acetate pH 5.5 buffer was made from sodium acetate trihydrate (2.72 g) made up to 9.5 mL with water, then titrated to pH 5.5 with acetic acid, and finally made up to 10 mL. NDS buffer was made from acetate solution (3.934 mL), sodium nitrite (380 mg, 5.5 mmol) and water (3.934 mL), then adjusted to pH 5.5 with 10 M sodium hydroxide (˜1 drop required). 2-chloro-2′-deoxyadenosine (0.5 mg), 2-fluoro-2′-deoxyadenosine (0.5 mg) and 2′-deoxyadenosine (0.5 mg) as a control were respectively placed in three vials and each dissolved in 1 mL solution of NDS buffer. The stability was monitored at intervals using LC/MS.

### Results

2-fluoro-2′-deoxyadenosine (2F-dA) was unreacted after 120 h incubation in NDS buffer
2-chloro-2′-deoxyadenosine (2Cl-dA) and 2′-deoxyadenosine (dA) react in the NDS buffer giving rise to 1 product, respectively.
Products of NDS reaction were differentiated and identified by LC/MS analysis and assigned to be deaminated nucleosides: 2-chloro-2′-deoxyinosine (2Cl-dl) and 2′-deoxyinosine (dl)
2-chloro-2′-deoxyadenosine (2Cl-dA) undergoes deamination in NDS buffer 5.5× slower than 2′-dA under the same conditions (Table 1/FIG. 6).
a) Stability of 2-fluoro-2′-deoxyadenosine incubated in NDS buffer for 24 h
   (UV peaks: at 1.265 min is related to NDS buffer and at 5.491 min corresponds to 2F-dA) (FIG. 3).
b) Stability of 2-fluoro-2′-deoxyadenosine incubated in NDS buffer for 48 h
   (UV peaks: at 1.265 min is related to NDS buffer and at 5.493 min corresponds to 2F-dA) (FIG. 3).
c) Stability of 2-fluoro-2′-deoxyadenosine incubated in NDS buffer for 120 h
   (UV peaks: at 1.265 min is related to NDS buffer and at 5.477 min corresponds to 2F-dA) (FIG. 3).
d) Stability of 2-chloro-2′-deoxyadenosine incubated in NDS buffer for 24 h
   (UV peaks: at 5.927 min corresponds to 2Cl-dA and at 5.096 min to 2CI-dl) (FIG. 4).
e) Stability of 2-chloro-2′-deoxyadenosine incubated in NDS buffer for 48 h
   (UV peaks: at 5.914 min corresponds to 2Cl-dA and at 5.081 min to 2CI-dl) (FIG. 4).
f) Stability of 2-chloro-2′-deoxyadenosine incubated in NDS buffer for 120 h
   (UV peaks: at 5.913 min corresponds to 2Cl-dA and at 5.084 min to 2CI-dl) (FIG. 4).
g) Stability of 2′-deoxyadenosine incubated in NDS buffer for 24 h
   (UV peaks: at 1.340 min is related to NDS buffer, at 4.265 min corresponds to 2′-dl and at 4.992 min to 2′-dA) (FIG. 5).
h) Stability of 2′-deoxyadenosine incubated in NDS buffer for 48 h
   (UV peaks: at 4.211 min corresponds to 2′-dl and at 4.971 min to 2′-dA) (FIG. 5).
i) Stability of 2′-deoxyadenosine incubated in NDS buffer for 120 h
   (UV peaks: at 4.214 min corresponds to 2′-dl and at 4.964 min to 2′-dA) (FIG. 5).

TABLE 1
Remaining nucleoside data for 2-chloro-2′-deoxyadenosine
(2-Cl-dA) vs. 2′-deoxyadenosine (dA) in NDS buffer
(displayed graphically in FIG. 6).

| Data | time | 2- | | In 2- | In |
|---|---|---|---|---|---|
| point | [h] | Cl-dA | dA | Cl-dA | dA |
| 1 | 24 | 99.42 | 96.66 | 4.599353301 | 4.571199666 |
| 2 | 48 | 98.66 | 92.85 | 4.591679596 | 4.530985288 |
| 3 | 120 | 96.54 | 82.77 | 4.56995743 | 4.416065677 |

### Synthesis of Reversible Terminator of 2-chloro-2′-deoxyadenosine

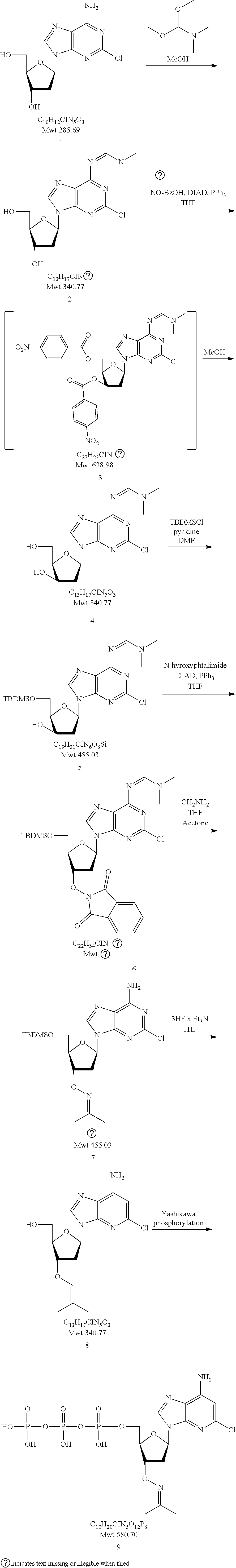

### Synthesis of N2-((Dimethylamino)methylene)-2-chloro-2′-deoxyadenosine (2)

A 50 mL round-bottomed flask equipped with a magnetic stir bar was consecutively charged with commercial 2-chloro-2′-deoxyadenosine (1) (0.95 g, 0.0033 mol), anhydrous DMF (8 mL) and N,N-dimethylformamide dimethyl acetal (2.214 mL, 1.98 g, 0.0166 mol, 5 equiv). The resultant suspension was vigorously stirred at room temperature. After 2 h reaction mixture was evaporated to dryness and lyophilised from water. The product was obtained as a pale yellow solid (1.1 g, 98%).

1H NMR (DMSO, 400 MHz) δ 8.97 (s, 1H, CH of imine), 8.54 (s, 1H, H8), 6.36 (dd, 1H, H-1′), 5.41 (br s, 1H, 5′-OH), 5.04 (br s, 1H, 3′-OH), 4.46-4.45 (m, 1H, H3′), 3.90-3.92 (m, 1H, H4′), 3.56-3.65 (m, 2H, H5′, H5″), 3.25, 3.29 (2×s, 6H, 2× —CH₃), 2.75-2.79 (m, 1H, H2′), 2.37-2.33 (m, 1H, H2″)

### Synthesis of N2-((Dimethylamino)methylene)-xylo-2-chloro-2′-deoxyadenosine (3)

N6-((Dimethylamino)methylene)-2-chloro-2′-deoxyadenosine (2) (1.05g, 0.0031 mol), 4-NO₂-BzOH (1.54 g, 0.0092 mol), TPP (3.23 g, 0.0123 mol) was placed into round bottom flask and anhydrous THF (15 mL) was added. To a stirred clear solution DIAD (2.43 mL, 2.49 g, 0.0123 mol) was added drop by drop over 5 min, maintaining the temperature at 15° C. and then resultant mixture was allowed to stir at room temperature. After 90 min h LC/MS revealed total consumption of starting material and formation of intermediate 3a. Reaction was quenched by the addition of MeOH (5 mL) which concomitantly caused exothermically catalysed deprotection of benzoate esters and then mixture was evaporated down to yellow oil. The residue was treated with 30 mL of isopropanol and stirred for 30 min when yellowish precipitate formed. The solid was filtered off solid and washed with iPrOH (1 mL). The product was obtained as a pale yellow solid (0.7 g, 66%).

1H NMR (DMSO, 400 MHz) δ 8.89 (s, 1H, CH of imine), 8.45 (s, 1H, H8), 6.35 (dd, 1H, H-1′), 5.43 (br s, 1H, 5′-OH), 4.70 (brS, 1H, 3′-OH), 4.32-4.36 (m, 1H, H3′), 3.92-3.95 (m, 1H, H4′), 3.69-3.755 (m, 1H, H5′), 3.55-3.65 (m, 1H, H5″) 3.15, 3.24 (2× s, 6H, 2× —CH₃), 2.70-2.80 (m, 1H, H2′), 2.25-2.30 (m, 1H, H2″)

### Synthesis of 5′-O-(tert-Butyldimethylsilyl)-N6-((Dimethylamino)methylene)-xylo-2-chloro-2′-deoxyadenosine (4)

N6-((Dimethylamino)methylene)-xylo-2-chloro-2′-deoxyadenosine (3) (0.6 g, 0.0018 mol) was dried under high vacuum for 2 h and anhydrous pyridine (0.38 mL) and DMF (5 mL) were added. To a stirred suspension, mixture of TBDMSCI (0.32 g, 0.0021 mol) in DMF (1.2 mL) was added dropwise over 5 mins at rt. The resultant solution was allowed to stir at rt for 30 min. Reaction was quenched by addition of cold water (40 mL) when white precipitate formed and the suspension was allowed to stir at rt for 1 h. Precipitate was collected by filtration and washed with water (3×5 mL). The product was obtained as a white solid (0.75 g, 94%).

1H NMR (DMSO, 400 MHz) δ 8.85 (s, 1H, CH of imine), 8.40 (s, 1H, H8), 6.25 (dd, 1H, H-1′), 5.48 (br S, 1H, 3′-OH), 4.32-4.36 (m, 1H, H3′), 3.93-3.96 (m, 2H, H4′, H5′), 3.72-3.79 (m, 1H, H5″) 3.22, 3.31 (2× s, 6H, 2× —CH₃), 2.68-2.78 (m, 1H, H2′), 2.24-2.27 (m, 1H, H2″), 0.83 (s, 9H, Si-C(CH₃)₃), −0.01 (s, 6H, Si(CH₃)₂)

### Synthesis of 3′-O-phthalimido-5′-O-tert-Butyldimethylsilyl-N6-((Dimethylamino)methylene)-2-chloro-2′-deoxyadenosine (5)

5′-O-(tert-Butyldimethylsilyl)-N6-((Dimethylamino)methylene)-xylo-2-chloro-T-deoxyadenosine (4) (0.62 g, 0.0014 mol), triphenylphosphine (0.68 g, 0.0026 mol) and N-hydroxyphthalimide (0.42 g, 0.0026 mol) were dissolved in anhydrous THF (5 mL) to get clear yellowish solution and the flask was immersed in an water-ice bath at 15° C. To a resultant mixture DIAD (0.51 mL, 0.52 g, 0.0026 mol) was added dropwise over 5 min, maintaining temperature of the reaction at 15° C. Upon completion of the addition the mixture turned yellow-orange and the flask was removed from the bath and the solution was allowed to stir at room temperature. After 1 h the reaction was quenched with MeOH (1 mL) and concentrated down using a rotary evaporator. The gummy orange residue was dissolved in toluene (4 mL) and stirred overnight. The precipitate was filtered off and washed with toluene (0.5 mL). The obtained solid was analysed to be a pure product. The liquor containing the product was concentrated and residue was subjected to purifications using precipitation with a mixture of 30% hexane in ethyl acetate. The product was obtained as a white solid (0.6 g, 73%).

1H NMR (DMSO, 400 MHz) δ 8.92 (s, 1H, CH of imine), 8.56 (s, 1H, H8), 8.92-8.98 (m, 4H, phthalimide group), 6.55 (dd, 1H, H-1′), 5.12-5.15 (m, 1H, H3′), 4.37-4.41 (m, 1H, H4′), 3.83-3.88 (m, 1H, H5′), 3.74-3.80 (m, 1H, H5″), 3.26, 3.18 (2× s, 6H, 2× —CH₃), 3.10-3.18 (m, 1H, H2′), 2.85-2.91 (m, 1H, H2″), 0.79 (s, 9H, Si—C(CH₃)₃), −0.01, 0.05 (2×s, 6H, Si(CH₃)₂)

### Synthesis of 3′-O-(N-acetone oxime)-5′-O-tert-Butyldimethylsilyl-2-chloro-2′-deoxy adenosine (6)

3′-O-phthalimido-5′-O-tert-Butyldimethylsilyl-thymidine (5) (0.54 g, 0.89 mmol) was dissolved in MeOH (0.3 mL) and 33% ethanolic solution of methylamine (1.3 mL, 8.99 mmol) was added. After 3 h LC/MS revealed total consumption of starting material and formation of aminooxy intermediate. Reaction mixture was placed in a water bath (22° C.) and acetone (0.66 mL, 0.89 mmol) was slowly added. The reaction mixture was allowed to stir for the next 30 min and diluted with ethyl acetate (10 mL) followed by extraction with a saturated solution of NaHCO₃ (2×2 mL) and water (3×2 mL). Organic phase was dried over MgSO₄, filtered and evaporated to dryness. The purity of the obtained product was sufficient to use it in the next step without preparative purification. The product was obtained as a white solid (0.35 g, 85%).

1H NMR (DMSO, 400 MHz) δ 8.29 (s, 1H, H8), 7.84 (br s, 2H, —NH₂), 6.24 (dd, 1H, H-1′), 4.81-4.77 (m, 1H, H3′), 4.08-4.12 (m, 1H, H4′), 3.82-3.88 (m, 1H, H5′), 3.71-3.76 (m, 1H, H5″), 2.78-2.85 (m, 1H, H2′), 2.52-2.58 (m, 1H, H2″), 1.83 (s, 6H, 2× —CH₃ of oxime), 0.81 (s, 9H, Si—C(CH₃)₃), −0.01, 0.04 (2×s, 6H, Si(CH₃)₂)

### Synthesis of 3′-O-(N-Acetone oxime)-2-chloro-2′-deoxyadenosine (7)

3′-O-(N-acetone oxime)-5′-O-tert-Butyldimethylsilyl-2-chloro-2′-deoxyadenosine (6) (0.28 g, 0.6 mmol) was dissolved in anhydrous THF (0.8 mL) and triethylamine trihydrofluoride (0.3 mL, 1.8 mmol) was added. Reaction mixture was stirred for 3 at room temperature and then ethoxytrimethylsilane (2 mL) and hexane (3 mL) were added when precipitate formed. The precipitate was collected by filtration and the solid was washed with hexane (2×1 mL). Product was obtained as a white solid (0.20 g, 95%).

1H NMR (DMSO, 400 MHz) δ 8.23 (s, 1H, H8), 7.73 (br s, 2H, —NH2), 6.00-6.13 (m, 1H, H-1′) 4.90-5.10 (m, 1H, 5′OH), 4.60-4.70 (m, 1H, H3′), 3.90-4.10 (m, 1H, H4′), 3.38-3.53 (m, 2H, H5′, H5″), 2.55-2.70 (m, 2H, H2′, H2″), 1.70 (s, 6H, 2× —CH₃ of oxime)

### Synthesis of 3′-O-(N-Acetone oxime)-2-chloro-2′-deoxyadenosine triphosphate (8)

A lyophilised 3′-O-(N-Acetone oxime)-2-chloro-2′-deoxyadenosine (7) (0.05 g, 0.147 mmol) was dissolved in trimethyl phosphate (0.5 mL) and tributylamine (0.08 mL, 0.06g, 0.32 mmol). The flask was placed in an ice-water bath at 0° C. and phosphorus oxychloride (0.015 mL, 0.025 g, 0.16 mmol) was added in one portion and the mixture was stirred for 10 min. Additional portion of phosphorus oxychloride (0.015 mL, 0.025 g, 0.16 mmol) was added to the reaction mixture and was stirred for next 30 minutes. A solution of tributylammonium pyrophosphate (0.161 g, 0.29 mmol), tributylamine (0.21 mL, 0.163 g, 0.88 mmol) in acetonitrile (0.5mL) was added to the reaction and kept stirring for 30 min. The reaction was then quenched by addition of a 10 mM TEAB buffer, pH 7.5 (2 mL) and stirred form 15 min. The mixture was evaporated to gummy oil and the crude triphosphate was subjected to purification by reverse phase C18-HPLC using preparative Phenomenex Kinetex C18 column (30×250 mm, 5 mm) (gradient elution at a flow rate of 25 mL/min with 100 mM Triethylammonium bicarbonate pH 7.5 (A) and acetonitrile (B), 2% B from 0 to 2 min, 2% to 25% B over 20 min, 25% B from 22 to 27 min, 25% to 2% B from 27 to 30 min, 2% B for 5 min). Fractions containing isolated triphosphate were pooled, evaporated to dryness, co-evaporated with methanol (3×10 mL) and lyophilised from water. The semi-purified triphosphate triethylamine salt was then purified by AEX-HPLC on Source15Q column (gradient elution at a flow rate of 35 mL/min with 10 mM Triethylammonium bicarbonate pH 7.5 (A) and 1M Triethylammonium bicarbonate pH 7.5 (B), 0% to 100% B over 32 min, 100% B from 32 to 34 min, 100% to 0% B from 34 to 36 min, 0% B for 10 min). Fractions containing isolated triphosphate were pooled, evaporated to dryness, co-evaporated with methanol (3×30 mL) and lyophilised from water. 3′-O-(N-Acetone oxime)-2-chloro-2′-deoxyadenosine triphosphate in the form of triethylamine salt was obtained as a white solid and the yield was determined by UV (260 nm, ext. coeff.=15400 Lmol-1cm-1) to be 47 umoles, ˜20%. 1H NMR (DMSO, 400 MHz) δ 8.37 (s, 1H, H8), 6.28 dd, 1H, H1′), 4.93-4.96 (m, 1H, H3′), 4.35-4.40 (m, 1H, H4′), 4.00-4.15 (m, 2H, H5′, H5″), 3.33 (q, 18H, CH₂ of TEA counterion) 2.59-2.72 (m, 2H, H2′, H2″), 1.80. 1.85 (s, 6H, 2× —CH₃ of oxime), 1.15 (t, 27H, CH₃ of TEA counterion)

31P NMR (D₂O, 400 MHz) δ −10.93 (m, 1P), −11.50 (d, 1P), −23.16 (m, 1P)

### Synthesis of Reversible Terminator of 2-fluoro-2′-deoxyadenosine

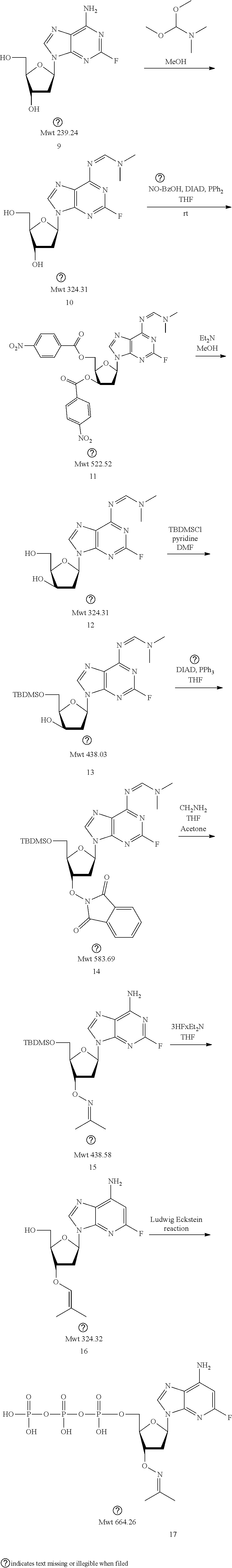

### Synthesis of N2-((Dimethylamino)methylene)-2-fluoro-2′-deoxyadenosine (10)

A 100 mL round-bottomed flask equipped with a magnetic stir bar was consecutively charged with commercial 2-fluoro-2′-deoxyadenosine (9) (1.00 g, 0.0037 mol), anhydrous MeOH (12 mL) and N,N-dimethylformamide dimethyl acetal (2.47 mL, 2.21 g, 0.0186 mol, 5 equiv). The reaction was allowed to stir at room temperature. After 1 h reaction mixture was evaporated to dryness and lyophilised from water. The product was obtained as a pale yellow solid (1.18 g, 98%).

1H NMR (DMSO, 400 MHz) 8.92 (s, 1H, CH of imine), 8.43 (s, 1H, H8), 6.29 (dd, 1H, H-1′), 5.35 (, 1H, 3′-OH), 4.96 (t, 1H, 5′-OH), 4.38-4.43 (m, 1H, H3′), 3.85-3.89 (m, 1H, H4′), 3.46-3.63 (m, 2H, H5′, H5″), 3.15, 3.26 (2×s, 6H, 2× —CH₃), 2.66-2.75 (m, 1H, H2′), 2.28-2.31 (m, 1H, H2″)

19F NMR (DMSO, 400 MHz) δ −51.66 (s, 1F)

### Synthesis of N2-((Dimethylamino)methylene)-3′,5′-di-(4-nitrobenzoate)-xylo-2-fluoro-2′-deoxyadenosine (11)

N2-((Dimethylamino)methylene)-2-fluoro-2′-deoxyadenosine (10) (1.15 g, 3.55 mmol), 4-nitrobenzoic acid (1.778 g, 10.64 mmol) and triphenylphosphine (3.72 g, 14.18 mmol) were placed in a flask, dissolved in anhydrous THF (15 mL) and immersed in water bath at 22° C. Diisopropyl azobisdicarboxylate (DIAD) (2.79 mL, 14.18 mmol) was slowly added to the mixture over 15 minutes. The temperature was controlled and maintained in the range 22-24° C. After the addition of DIAD, the reaction turned green. The solution was stirred for 1 h at room temperature when the reaction turned yellow. Isopropanol was added (40 mL) and a precipitate formed. The yellow suspension was stirred for 2 hours, then filtered and the solid was washed with isopropanol (3×10 mL)). The product was obtained as a pale yellow solid (1.85 g, 84%).

1H NMR (DMSO, 400 MHz) δ 8.84 (s, 1H, H2), 8.45 (s, 1H, H8), 8.32-8.25-8.50 (m, 4H, 4-NO₂-Bz group), 8.04-8.09 (m, 4H, 4-NO₂-Bz group) 6.39-6.43 (m, 1H, H-1′), 5.85-5.9 (m, 1H, H3′), 4.70-4.83 (m, 3H, H4′,H5′,H5″), 3.14, 3.23 (2×s, 6H, 2x -CH₃) 3.06-3.12 (m, 2H, H2′,H2″)

19F NMR (DMSO, 400 MHz) δ −51.52 (s, 1F)

### Synthesis of N2-((Dimethylamino)methylene)-xylo-2-fluoro-2′-deoxyadenosine (12)

N2-((Dimethylamino)methylene)-3′,5′-di-(4-nitrobenzoate)-xylo-2-fluoro-2′-deoxyadenosine (11) (1.70 g, 2.73 mmol) was suspended in dry methanol (13.68 mL). Triethylamine (1.52 mL, 10.9 mmol) was added and the reaction was heated to reflux for 30 min when a clear yellow solution resulted. The solution was left to cool down at room temperature when white precipitate formed and hexane was added (1 mL). The white precipitate was collected by filtration and washed with hexane (1 mL). The solid was suspended in water (30 mL) and stirred for 30 min. The white solid was filtered off and washed water (2×5 mL). The liquor was frozen and lyophilized to give pure xylo-2-fluoro-2′-deoxyadenosine as a white solid (0.8 g, 91%).

1H NMR (DMSO, 400 MHz) 8.92 (s, 1H, CH of imine), 8.44 (s, 1H, H8), 6.24 (dd, 1H, H-1′), 5.43 (d, 1H, 3′-OH), 4.57 (t, 1H, 5′-OH), 4.35-4.39 (m, 1H, H3′), 3.91-3.96 (m, 1H, H4′), 3.71-3.76 (m, 1H, H5′), 3.57-3.64 (m, 1H, H5″) 3.15, 3.24 (2×s, 6H, 2× —CH₃), 2.71-2.77 (m, 1H, H2′), 2.25-2.27 (m, 1H, H2″)

19F NMR (DMSO, 400 MHz) δ −51.66 (s, 1F)

19F NMR (DMSO, 400 MHz) δ −51.90 (s, 1F)

### Synthesis of N2-((Dimethylamino)methylene)-5′-O-(tert-Butyldimethylsilyl)-xylo-2-fluoro-2′-deoxyadenosine (13)

N2-((Dimethylamino)methylene)-xylo-2-fluoro-2′-deoxyadenosine (12) (0.68 g, 2.1 mmol) was dissolved anhydrous pyridine (0.69 mL) and anhydrous DMF (3 mL). To a stirred solution, mixture of TBDMSCI (0.38 g, 0.4 mmol) in DMF (1 mL) was added dropwise over 5 min at room temperature. The resultant solution was allowed to stir at rt for 15 min. Reaction was quenched by addition of cold water (15 mL) when white precipitate formed and the suspension was allowed to stir for 1 h. Precipitate was collected by filtration and washed with water (3×2 mL). The product was obtained as a white solid (0.9 g, 98%).

1H NMR (DMSO, 400 MHz) δ 8.89 (s, 1H, CH of imine), 8.40 (s, 1H, H8), 6.2-6.25 (dd, 1H, H-1′), 5.47 (d, 1H, 3′-OH), 4.35-4.39 (m, 1H, H3′), 3.90-3.98 (m, 2H, H4′,H5′), 3.72-3.79 (m, 1H, H5″) 3.15, 3.23 (s, 6H, 2× —CH₃), 2.68-2.75 (m, 1H, H2′), 2.23-2.28 (m, 1H, H2″), 0.82 (s, 9H, Si—C(CH₃)3), −0.01 (s, 6H, Si(CH₃)₂)

### Stability Studies of 8-aza-adenosine and 2′-deoxyadenosine in NDS Buffer

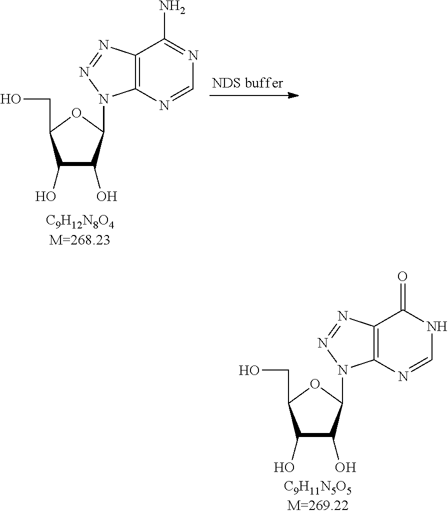

A solution of NDS made up from sodium nitrite, sodium acetate pH 5.5) and titrated to pH 5.5 with 10 M NaOH was obtained (2 mL).

1 mL aliquots of this solution were added to 8-aza-2′-dA (0.5 mg) and 2′-dA (0.5 mg, as a control to compare how additional N atom at 8 position of nucleobase affects reactivity NH₂—C6 group with NDS) and analysed at intervals using LC/MS.

| | | 8-aza-A | dA | | |
|---|---|---|---|---|---|
| | | (consump- | (consump- | | |
| | | tion of | tion of | | |
| Data | time | nucleoside, | nucleoside, | In 8- | In |
| point | [h] | peak area %) | peak area %) | aza-A | dA |
| 1 | 24 | 98.65 | 94.15 | 4.591578232 | 4.544889255 |
| 2 | 96 | 93.49 | 77.6 | 4.537854479 | 4.351567427 |
| 3 | 120 | 91.74 | 73 | 4.518958489 | 4.290459441 |
| 4 | 146 | 90 | 70 | 4.49980967 | 4.248495242 |

### Conclusion

1. 8-aza-A and dA react in the NDS buffer giving rise to 1 product, respectively.
2. Products of NDS reaction was identified by LC/MS analysis and assigned to be deaminated nucleosides: 8-aza-inosine and 2′-deoxyinosine
3. 8-aza-A undergoes deamination in NDS buffer 3.2× slower than 2′-dA under the same conditions

### BRIEF DESCRIPTION OF THE FIGURES

FIG. 1: LC trace of commercial 2-chloro-2′-deoxyadenosine and 2-chloro-2′-deoxyadenosine incubated in 1×ORS buffer for 24 h.

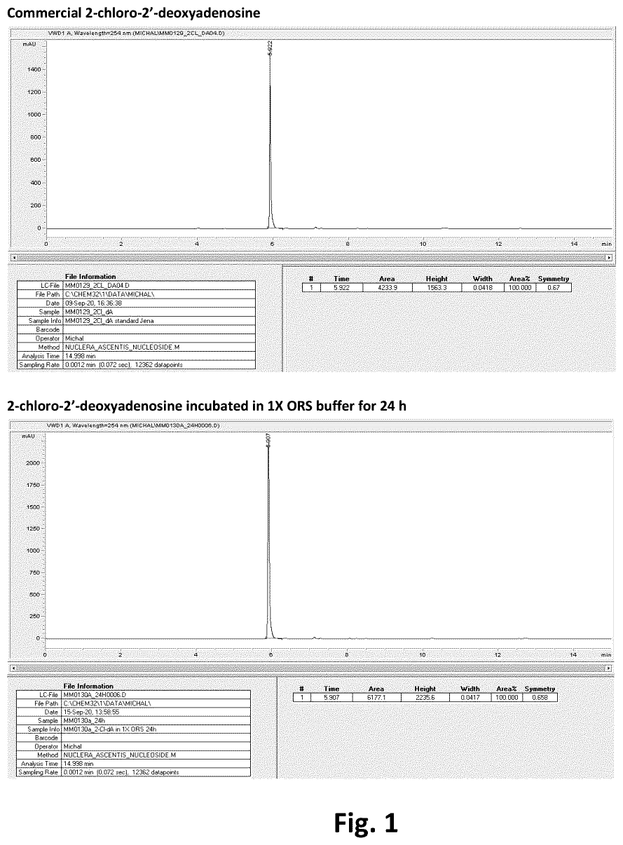

FIG. 2: LC trace of commercial 2-fluoro-2′-deoxyadenosine and 2-fluoro-2′-deoxyadenosine incubated in 1×ORS buffer for 24 h.

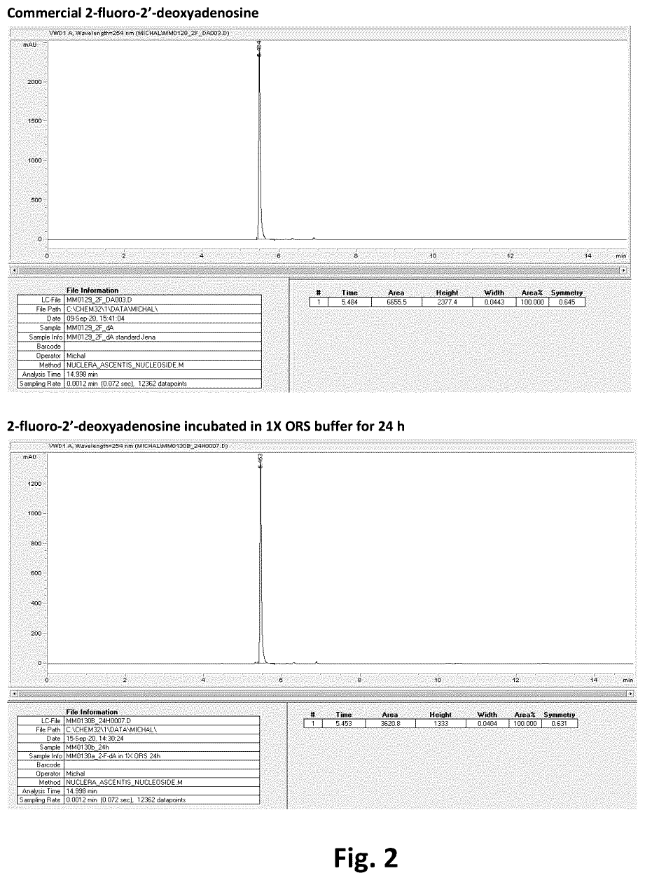

FIG. 3: LC trace of 2-fluoro-2′-deoxyadenosine incubated in NDS buffer for 24 h, 48 h and 120 h.

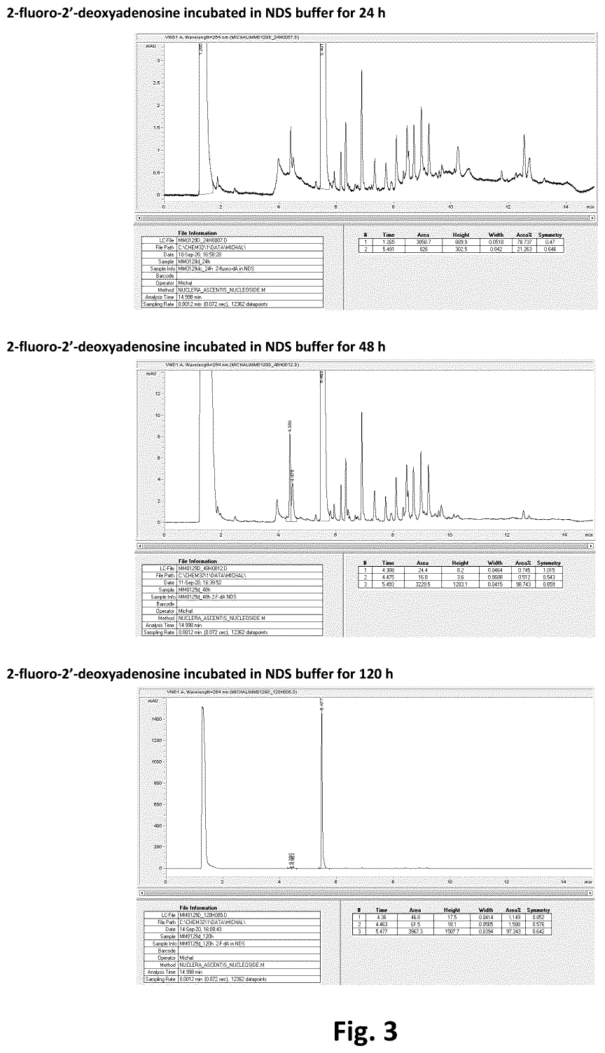

FIG. 4: LC trace of 2-chloro-2′-deoxyadenosine incubated in NDS buffer for 24 h, 48 h and 120 h.

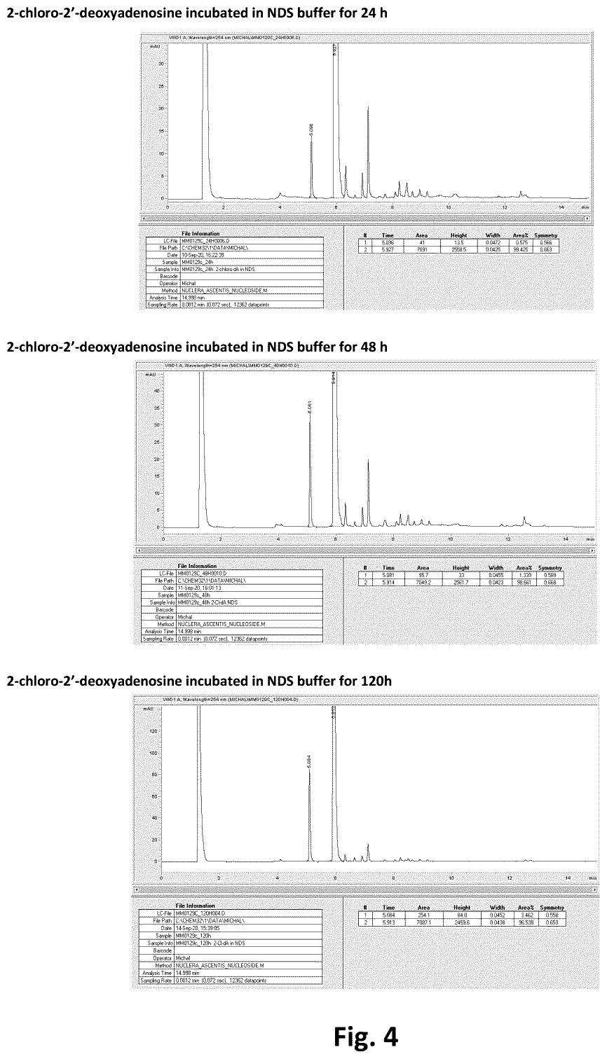

FIG. 5: LC trace of 2′-deoxyadenosine incubated in NDS buffer for 24 h, 48 h and 120 h.

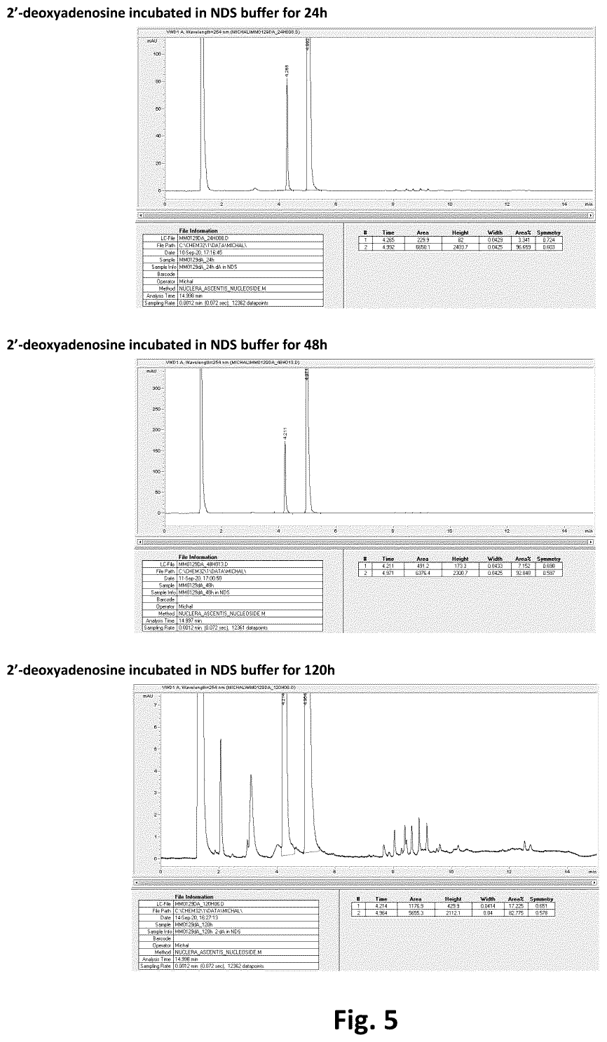

FIG. 6: Graphical representation of data showing the percentage remaining nucleoside starting material for 2-chloro-2′-deoxyadenosine (2-Cl-dA) vs. 2′-deoxyadenosine (dA) in NDS buffer.

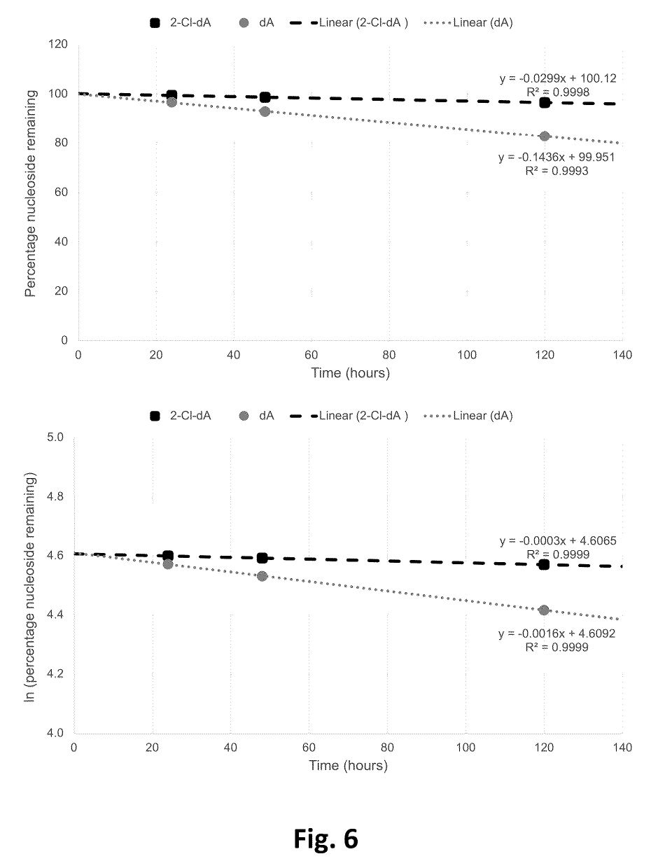

FIG. 7: Graphical representation of data showing the percentage remaining nucleoside starting material deoxyadenosine (8-aza-dA) vs. 2′-deoxyadenosine (dA) in NDS buffer.

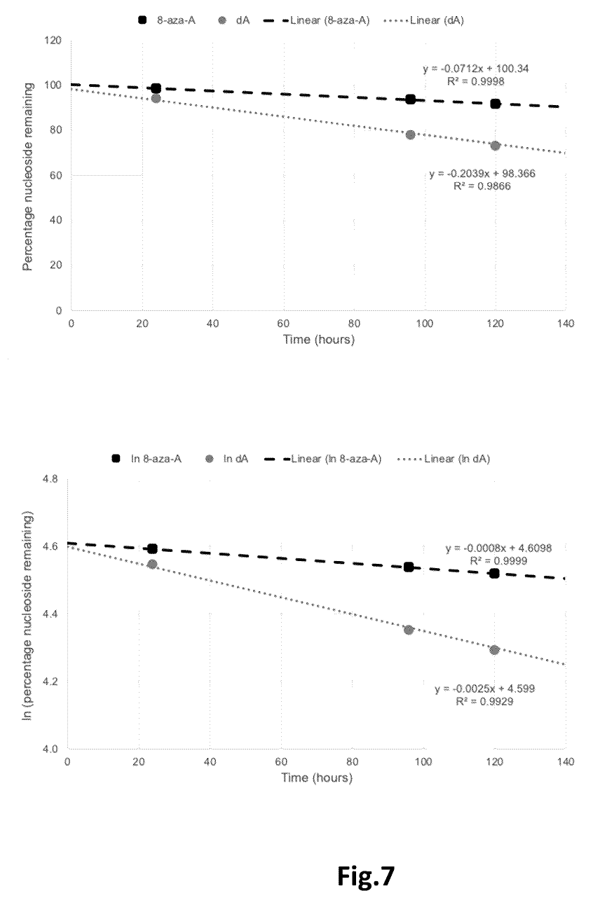

1. A compound according to Formula (1a) or (1b): 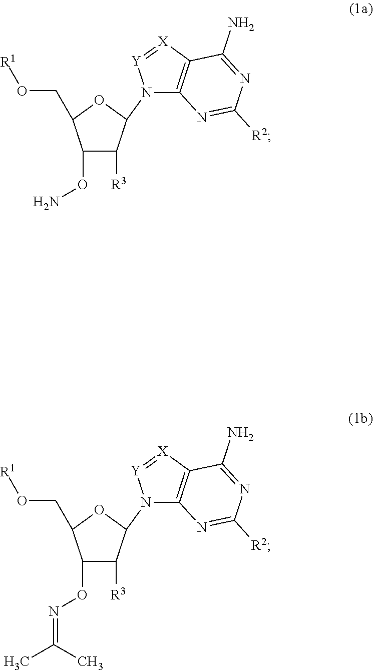
wherein,
R¹ is a phosphate or polyphosphate group or salt thereof, optionally containing one or more sulfur atoms;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo;
nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

2. The compound according to claim 1 which is a compound of Formula (2a) or (2b): 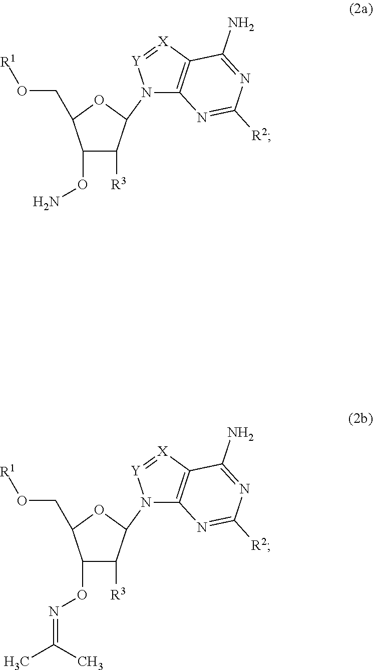
wherein,
R¹ is a phosphate or polyphosphate group or salt thereof, optionally containing one or more sulfur atoms;
R² is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe;
X is N, CH or CR⁷ where R⁷ is optionally substituted C₁₋₅ alkyl, optionally substituted C₁₋₅ alkenyl or optionally substituted C₁₋₅ alkynyl;
Y is CH or N; and
wherein R⁴ and R⁵ are independently selected from H and C₁₋₆ alkyl optionally substituted with OH or halo atoms.

3. The compound according to claim 2, wherein R² is halo, halomethyl, dihalomethyl or trihalomethyl.

4. The compound according to claim 3, wherein R² is F or Cl.

5. The compound according to claim 1 which is a compound of Formula (3a) or (3b): 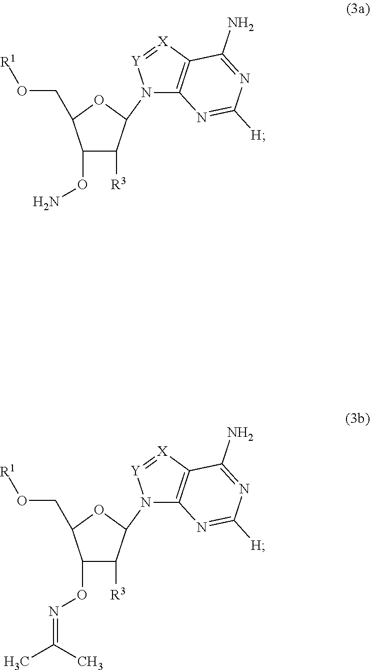
wherein,
R¹ is a phosphate or polyphosphate group or salt thereof, optionally containing one or more sulfur atoms;
R³ is selected from H, OH, F, OCH₃, or OCH₂CH₂OMe; and
X is N or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

6. The compound according to any one of claims 1 to 5 wherein X is CR⁸ and R⁸ is F, Cl or CF₃.

7. The compound according to any one of claims 1 to 5 wherein X is N and Y is N.

8. The compound according to any one of claims 1 to 7, wherein R¹ is —(PO₃)⁻ₓ(PO₂S)⁻_{y}(PO₃)⁻_{z} where x, y and z are independently 0-5 and x+y+z is 1-5.

9. The compound according to any one of claims 1 to 7, wherein R¹ is a monophosphate, diphosphate, triphosphate, tetraphosphate, pentaphosphate, or (alpha-thio)triphosphate group.

10. The compound according to any one of claims 1 to 9, wherein R¹ is a triphosphate group.

11. The compound according to any one of claims 1 to 10, wherein R³ is H.

12. The compound according to claims which is selected from the group consisting of: 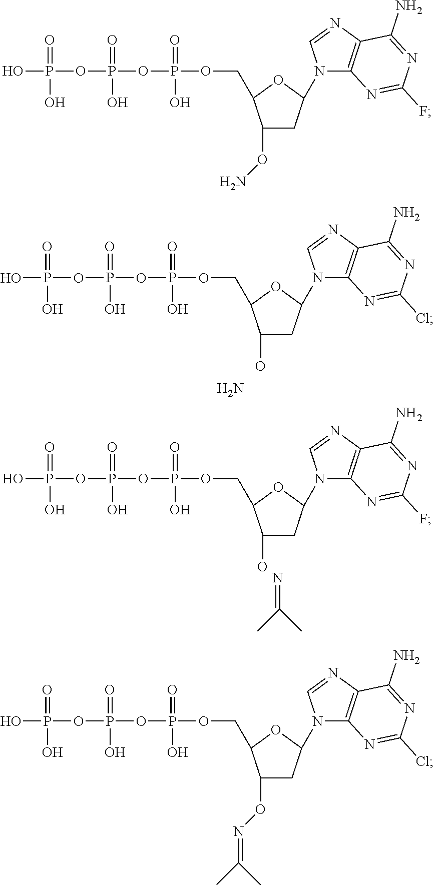 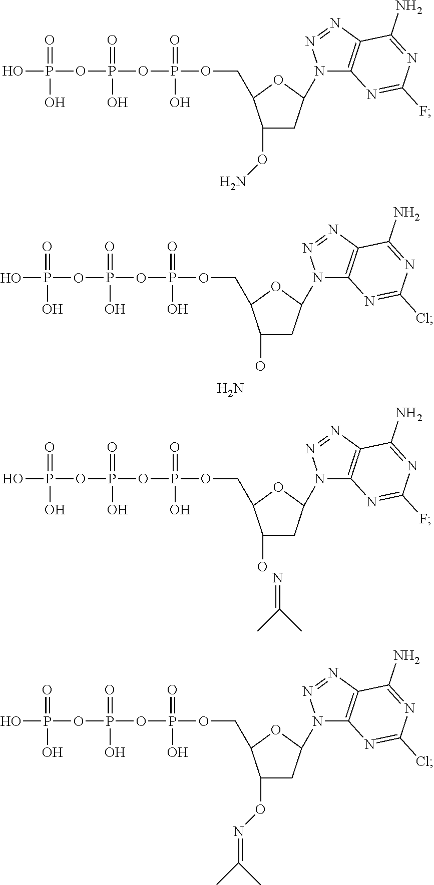 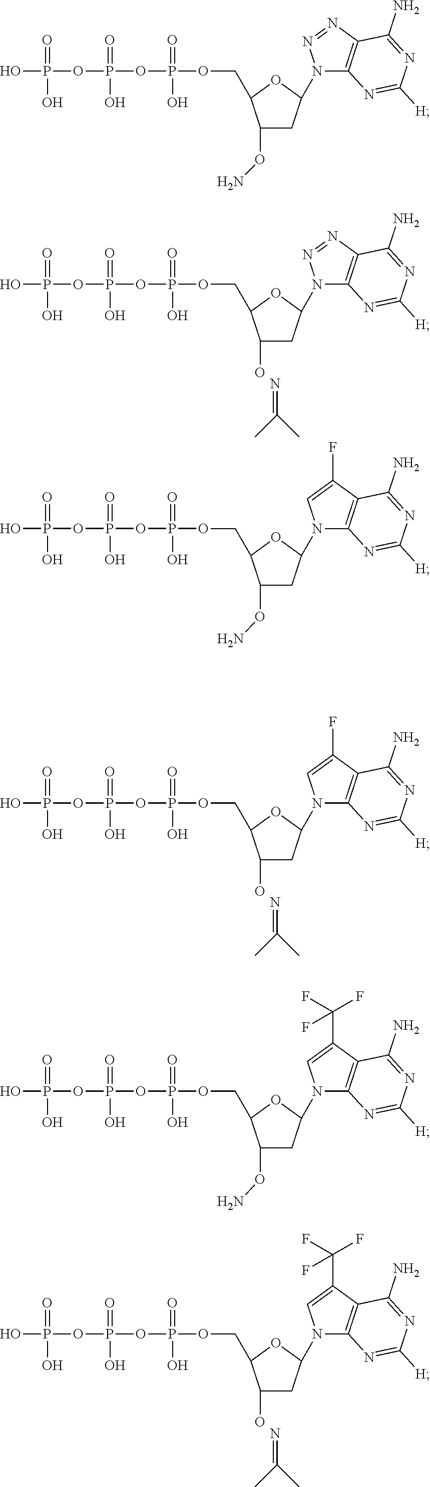
or a salt thereof.

13. A method of nucleic acid synthesis comprising reacting a compound according to any one of claims 1 to 12 with an oligonucleotide in the presence of a polymerase or terminal deoxynucleotidyl transferase (TdT) enzyme and treating the extended oligonucleotide with a nitrite salt.

14. The method according to claim 13, wherein the oligonucleotide sequence is a solid-supported oligonucleotide sequence.

15. The method according to claim 13 or claim 14, wherein the nitrite salt is sodium nitrite.

16. A method of synthesizing a compound according to formula (1a), (2a) or (3a) by treating the compounds of Formula (1b), (2b) or (3b) with an aminooxy compound.

17. The method according to claim 16 wherein the aminooxy compound is hydroxylamine, methoxylamine or ethoxylamine.

18. A kit comprising:
a. a compound according to any one of claims 1 to 12;
b. a terminal deoxynucleotidyl transferase (TdT) enzyme; and
c. a nitrite salt.

19. An oligonucleotide according to Formula (1a) or (1b): 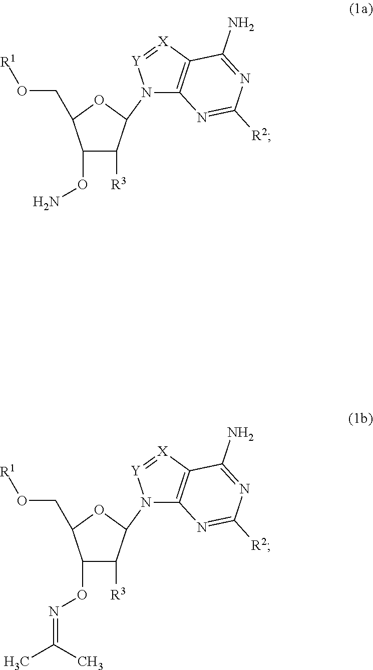
wherein,
R¹ is an oligonucleotide;
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe; and
X is N, CH, CR⁷ where R⁷ is optionally substituted C₁₋₅ alkyl, optionally substituted C₁₋₅ alkenyl or optionally substituted C₁₋₅ alkynyl, or CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵; and
Y is CH or N; and
either
X is N and Y is N, or
X is CR⁸ where R⁸ is an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵, and/or
R² is H or an electron withdrawing group (EWG) selected from the group consisting of: halo; nitro, nitrosyl, nitrile; halomethyl, dihalomethyl, trihalomethyl; C≡CR⁴; SOR⁴; SO₂R⁴; SO₃R⁴; COR⁴; CO₂R⁴; CONR⁴R⁵;
wherein each R⁴ and R⁵ are independently selected from H and C₁₋₆ alkyl optionally substituted with OH or halo atoms.

20. The oligonucleotide according to claim 19 wherein R² is F or Cl.

21. The oligonucleotide according to any one of claim 19 or 20 wherein R³ is H.

22. The oligonucleotide according to any one of claims 19 to 21 wherein X is N and Y is N.

23. The oligonucleotide according to any one of claims 19 to 21 wherein X is CFe and Fe is F, Cl or CF₃.
